(19) United States
(12) Patent Application Publication
(10) Pub. No.: US 2024/0318206 A1
(43) Pub. Date: Sep. 26, 2024

(54) TRANSCRIPTION TERMINATOR BIOPARTS BASED ON 3'-UNTRANSLATED REGION (UTR) AND A METABOLIC ENGINEERING METHOD THEREOF

(71) Applicant: KOREA ADVANCED INSTITUTE OF SCIENCE AND TECHNOLOGY, Daejeon (KR)

(72) Inventors: Byung-Kwan CHO, Daejeon (KR); Donghui CHOE, Daejeon (KR); Kang San KIM, Daejeon (KR)

(73) Assignee: KOREA ADVANCED INSTITUTE OF SCIENCE AND TECHNOLOGY, Daejeon (KR)

(21) Appl. No.: 18/585,375

(22) Filed: Feb. 23, 2024

(30) Foreign Application Priority Data
Feb. 24, 2023 (KR) 10-2023-0025311

(51) Int. Cl. C12P 7/04; C12N 9/04; C12N 9/10; C12N 9/12; C12N 9/88; C12N 9/90; C12N 15/70; C12P 7/22

(52) U.S. Cl. CPC C12P 7/04; C12N 9/0006; C12N 9/1022; C12N 9/1205; C12N 9/88; C12N 9/90; C12N 15/70; C12P 7/22; C12Y 101/01004; C12Y 202/01006; C12Y 207/01011; C12Y 401/01005; C12Y 505/01004; C12N 2800/101

(57) ABSTRACT

The present invention relates to a transcription regulatory biopart based on the 3′-untranslated region and a metabolic flux control method thereof.

### CROSS-REFERENCE TO RELATED APPLICATIONS

This application claims priority to Korean Patent Application No. 10-2023-0025311, filed on Feb. 24, 2023, and entitled “Transcription terminator bioparts based on 3′-untranslated region (UTR) and a metabolic engineering method thereof,” which is incorporated herewith in its entirety.

### TECHNICAL FIELD

The present invention relates to a transcription regulatory biopart based on the 3′-untranslated region and a metabolic flux control method thereof.

### BACKGROUND ART

Factors including promoters and 5′-untranslated regions (ribosome binding sites, etc.) are commonly used for gene expression and metabolic pathway control in microorganisms. Existing regulatory factors are all involved in gene transcription or translation initiation, where numerous additional factors are involved, and thus this results in considerable variations between experiments and necessitates expensive chemicals for regulation.

Additionally, when scaling up useful substance-producing microorganisms developed in the laboratory for actual industrial production, growth conditions and culture environment change, and consequently, the amount of substance production is significantly increased, which necessitates incidental optimization processes and strict control of raw materials added and culture conditions according to target compounds.

To solve these limitations, there is a demand for metabolic regulation technologies that exhibit low deviation between experiments and stability against changes in external environments, such as nutrients and culture conditions.

Meanwhile, transcriptional terminators serve to regulate the precise transcription of only desired genes when genetic information encoded by DNA is transcribed into RNA. Therefore, the present inventors developed a transcriptional terminator biopart usable in gene expression and metabolic pathway control in microorganisms and identified that the transcriptional terminator biopart can be used for metabolic pathway control of useful biocompounds.

### PRIOR ART DOCUMENTS

### Non-Patent Document

Nucleic Acids Res. 2017 Apr. 7; 45(6): 3487-3502.

### DISCLOSURE

### Technical Problem

An aspect of the present invention is to provide a nucleic acid construct including first and second genes, of which expressions are regulated by one promoter, wherein the first gene is located upstream of the second gene, the promoter is located upstream of the first gene, and an E. coli-derived Rho-independent terminator is located at the 3′-end of the first gene.

Another aspect of the present invention is to provide a microorganism including the nucleic acid construct.

Still another aspect of the present invention is to provide a method for producing a useful substance, the method including culturing the microorganism.

### Advantageous Effects

The transcriptional terminator biopart included in the 3′-UTR of the present invention can be used for gene expression and metabolic flux control in microorganisms as well as for metabolic pathway control of useful biocompounds.

### Technical Solution

The present invention will be specifically described as follows. Each description and exemplary embodiment disclosed herein may also be applied to other descriptions and exemplary embodiments. That is, all combinations of various elements disclosed herein fall within the scope of the present invention. Furthermore, the scope of the present invention is not limited by the specific description below.

Furthermore, a person skilled in the art will recognize or be able to ascertain, by using no more than routine experimentation, many equivalents to the specific embodiments of the present invention described herein. Furthermore, these equivalents are intended to be included in the present invention.

Furthermore, throughout the overall specification, many papers and patent documents are referenced and their citations are provided. The disclosures of cited papers and patent documents are entirely incorporated by reference herein, and the level of the technical field within which the present invention falls and details of the present invention are explained more clearly.

In accordance with an aspect of the present invention, there is provided a nucleic acid construct including an E. coli-derived Rho-independent terminator at the 3′-end of a gene. The 3′-end including the Rho-independent terminator is an untranslated region (3′-UTR).

As used herein, the term “nucleic acid construct” refers to a single- or double-stranded nucleic acid molecule, which includes one or more regulatory sequences and which is artificially synthesized, or manipulated to include a specific sequence in a manner that does not exist in nature, or isolated from nature. An example thereof may be an expression vector.

The nucleic acid construct provided in the present invention may include first and second genes having a polycistronic structure and include a Rho-independent terminator between the first and second genes.

Specifically, the nucleic acid construct provided in the present invention has a structure comprising first and second genes, of which expressions are regulated by one promoter, wherein the first gene is located upstream of the second gene, the promoter is located upstream of the first gene, and an E. coli-derived Rho-independent terminator is included at the 3′-end of the first gene.

In the present invention, the untranslated region (UTR) is directed to a region which is transcribed but is not translated into an amino acid sequence, or to a corresponding region in an RNA molecule, such as an mRNA molecule. The 3′-UTR is located downstream of the termination codon of a protein-encoding region, at the 3′-end of a gene.

In the present invention, the terminator is a nucleic acid sequence that is functionally linked to the end of a nucleic acid to be transcribed to be capable of stopping the transcription of the nucleic acid. The terminator is known to regulate the transcription levels by tuning transcription termination and mRNA 3′-end processing.

In the present invention, the Rho-independent terminator is distinguished from a Rho-dependent terminator, in which Rho protein is involved in transcription termination, and the termination is controlled by the RNA sequence of the Rho-independent terminator. The transcription termination involving such a Rho-independent terminator is also referred to as intrinsic termination. It is known that mRNA forms a stem-loop or hairpin structure due to the terminator sequence and this structure is involved in the separation of RNA polymerase from the DNA template strand to induce transcription termination.

As an example, the Rho-independent terminator of the present invention may be derived from E. coli.

For example, the Rho-independent terminator of the present invention may include a polynucleotide sequence selected from SEQ ID NOS: 1 to 12, or may essentially consist of or consist of the sequence.

The present inventors identified that various Rho-independent terminator sequences have different termination efficiencies and identified through examples that the terminator sequence variation can regulate expression levels of genes encoding respective proteins in the polycistronic structure. It was also identified that such a change of the 3′-UTR site does not increase noise compared to a method of manipulating 5′-UTR or trans-acting elements and can act regardless of host cells. It was also identified that the regulation of gene expression using the Rho-independent terminator sequence increased the yields of useful products, such as 2,3-BDO and myo-inositol (MI).

In the nucleic acid construct of the present invention, the first and second genes encode different proteins and are distinguished from each other due to the presence of a terminator downstream of the first gene.

In the nucleic acid construct of the present invention, the first or second gene may separately encode one polypeptide each, but is not limited thereto, and may encode two or more polypeptides.

In one embodiment, the first gene in the nucleic acid construct of the present invention may include polynucleotide sequences encoding acetolactate synthase (AlsS) and acetolactate decarboxylase (AlsD). In the foregoing embodiment, the second gene of the nucleic acid construct of the present invention may include a polynucleotide sequence encoding butanediol dehydrogenase (Bdh).

In any one of the foregoing embodiments, the nucleic acid construct of the present invention may include a structure of operatively linked promoter-alsS-alsD-Rho-independent terminator-bdh.

In any one of the foregoing embodiments, in the nucleic acid construct including a structure of promoter-alsS-alsD-Rho-independent terminator-bdh, the Rho-independent terminator may include a polynucleotide sequence selected from SEQ ID NOS: 1 to 8, or may essentially consist of or consist of the sequence.

In any one of the foregoing embodiments, in the nucleic acid construct including a structure of promoter-alsS-alsD-Rho-independent terminator-bdh, the Rho-independent terminator may include a polynucleotide sequence selected from SEQ ID NOS: 5 to 8, or may essentially consist of or consist of the sequence.

In any one of the foregoing embodiments, the RBS sequence may be included between alsS and alsD.

In one of the foregoing embodiments, the alsS gene may be derived from B. subtilis. As an example, the alsS gene may include the polynucleotide sequence of SEQ ID NO: 13, a degenerate sequence thereof, or a codon-optimized sequence thereof.

In one of the foregoing embodiments, the alsD gene may be derived from Aeromonas hydrophila. As an example, the alsD gene may include the polynucleotide sequence of SEQ ID NO: 14, a degenerate sequence thereof, or a codon-optimized sequence thereof.

In one of the foregoing embodiments, the bdh gene may be derived from Thermoanaerobacter brockii. As an example, the bdh gene may include the polynucleotide sequence of SEQ ID NO: 16, a degenerate sequence thereof, or a codon-optimized sequence thereof.

In any one of the foregoing embodiments, the nucleic acid construct including a structure of promoter-alsS-alsD-Rho-independent terminator-bdh can increase the microorganism's production of 2,3-butanediol (BDO) compared to microorganisms including a nucleic acid construct having no Rho-independent terminator.

In another embodiment, the first gene of the nucleic acid construct of the present invention may include a gene encoding MI-1-phosphate synthase (INO1). In the foregoing embodiment, the second gene of the nucleic acid construct of the present invention may include a gene encoding phosphofructokinase I (PfkA).

In any one of the foregoing embodiments, the nucleic acid construct of the present invention may include a structure of operatively linked promoter-ino1-Rho-independent terminator-pfkA.

In any one of the foregoing embodiments, in the nucleic acid construct including a structure of promoter-ino1-Rho-independent terminator-pfkA, the Rho-independent terminator may include a polynucleotide sequence selected from SEQ ID NOS: 1 to 4, or may essentially consist of or consist of the sequence.

In one of the foregoing embodiments, the ino1 gene may be derived from Saccharomyces cerevisiae CEN.PK. As an example, the ino1 gene may include the polynucleotide of SEQ ID NO: 15, a degenerate sequence thereof, or a codon-optimized sequence thereof.

In any one of the foregoing embodiments, the pfkA gene may be derived from E. coli. As an example, the pfkA gene may include the polynucleotide sequence of SEQ ID NO: 17, a degenerate sequence thereof, or a codon-optimized sequence thereof.

In any one of the foregoing embodiments, the nucleic acid construct including a structure of promoter-ino1-Rho-independent terminator-pfkA can increase the microorganism's production of myo-inositol (MI, also referred to as vitamin B8) compared to microorganisms including a nucleic acid construct having no Rho-independent terminator.

In accordance with another aspect of the present invention, there is provided a microorganism including the nucleic acid structure.

In an embodiment, the microorganism of the present invention may have a minimal genome.

In an embodiment, the microorganism of the present invention may be a wild-type microorganism.

In any one of the foregoing embodiments, the microorganism may be Escherichia coli. An example thereof may be MG1655 or eMS57.

In any one of the foregoing embodiments, the microorganism uses MG1655 as a parent strain thereof, and in the structure of promoter-ino1-Rho-independent terminator-pfkA, the Rho-independent terminator may include a sequence selected from SEQ ID NOS: 2 to 4.

In any one of the foregoing embodiments, the microorganism uses eMS57 as a parent strain thereof, and in the structure of promoter-ino1-Rho-independent terminator-pfkA, the Rho-independent terminator may include a sequence selected from SEQ ID NOS: 2 to 4 and 12.

In any one of the foregoing embodiments, the microorganism may further include an alteration of a metabolic pathway to increase the ability to produce a target product. For example, when the target product is myo-inositol, the microorganism may include an alteration, such as attenuation of glucose-6-phosphate dehydrogenase (zwf) or attenuation of glucose-6-phosphate isomerase (pgi). However, the alteration is not limited thereto.

In accordance with another aspect of the present invention, there is provided a method for producing a useful product, the method including culturing the microorganism.

In an embodiment, the useful product may be 2,3-butanediol (2,3-BDO). When the useful product is 2,3-butanediol, the inclusion of a specific Rho-independent terminator between the alsSD gene and the bdh gene can also increase the production of acetoin.

In an embodiment, the useful product may be myo-inositol.

### BRIEF DESCRIPTION OF DRAWINGS

FIG. 1: Discovery of diverse classes of transcript 3′-ends (T3PEs) in Escherichia coli using Term-Seq. FIG. 1A shows the conserved sequence motif of Rho-independent terminators upstream of the 407 transcript 3′-ends in the presence of a conserved sequence motif (RITs). FIG. 1B shows a predicted secondary structure of one of the RITs, T3PE-713, which includes a stable stem-loop structure. FIG. 1C shows the comparison of the predicted folding energies of the different RNA termini classes. Random indicates the folding energy of 1,000 random genomic positions. Dar indicates the folding energy of 1,095 previously reported T3PEs in Dar, D. and Sorek, R. (2018) High-resolution RNA 3′-ends mapping of bacterial Rho-dependent transcripts. Nucleic Acids Res., 46, 6797-6805. Three duplicated T3PEs were removed. Box limits, whiskers, and center lines indicate 1st and 3rd quartiles, 10th and 90th percentiles, and the median of the distribution, respectively. FIG. 1D shows that the Term-Seq results revealed the nucleotide-level footprint of ribosomal RNA and transfer RNA processing in the r/tRNA operon. The different RNase cleavage sites are marked by arrowheads. FIG. 1E shows the composition of the T3PE classes in this study and the comparison results with previous reports (Adams, P. P., Baniulyte, G., Esnault, C., Chegireddy, K., Singh, N., Monge, M., Dale, R. K., Storz, G. and Wade, J. T. (2021) Regulatory roles of Escherichia coli 5UTR and ORF-internal RNAs detected by 3 end mapping. Elife, 10, e62438. and Dar, D. and Sorek, R. (2018) High-resolution RNA 3′-ends mapping of bacterial Rho-dependent transcripts. Nucleic Acids Res., 46, 6797-6805). FIG. 1F shows the meta-analysis results of the RNA-Seq profile showing the RNA density near the T3PEs.

FIG. 2: Results of dual fluorescence reporter assay measurements of the transcription termination by upstream sequences of transcript 3′-ends (T3PEs). FIG. 2A shows the plasmid map of the dual reporter assay plasmid pDRA1. FIG. 2B shows that Escherichia coli harboring different pDRA1 plasmids displayed different red fluorescent protein (RFP) expressions when grown on agar media. Low RFP expression indicates strong termination strength. FIG. 2C shows that the monitoring the two different fluorescence signals revealed diverse termination activities of 150 T3PEs. Two signals were normalized by setting the green fluorescent protein (GFP) and RFP signals of pDRA1 to the same value. Error bars indicate the standard deviation of two biological replicate cultures. FIG. 2D shows that read-through distributions of the motif-less T3PEs revealed T3PEs differing significantly from RITs. Read-through indicates RFP intensity divided by GFP intensity. *p-value: 0.0478 (Welch's t-test). Box limits, whiskers, and center lines indicate 1st and 3rd quartiles, 10th and 90th percentiles, and the median of the distribution, respectively. Circles indicate individual T3PEs. Colored horizontal lines denote read-throughs of pDRA1_empty and pDRA1_rrnBT. FIGS. 2E to 2G show transcript profiles of T3PEs that are independent of canonical transcriptional termination. Transcripts with 3′-termini are products of (E) attenuation, (F) RNA stabilizing element, and (G) RNase III cleavage. Transcript 5′-end mapping supports the formation of an RNase III cleavage footprint. FIG. 2H shows the secondary structure of 5′-UTR of proP mRNA. Arrows indicate the RNase III cleavage sites discovered elsewhere (Lim, B. and Lee, K. (2015) Stability of the osmoregulated promoter-derived proP mRNA is post-transcriptionally regulated by RNase III in Escherichia coli. J. Bacteriol., 197, 1297-1305) and locations of T3PE-1523, which coincide with each other.

FIG. 3: Differential termination strengths of RITs at different transcription levels. FIG. 3A shows the design of the dual reporter assay plasmid, pDRA2, with transcription initiation levels regulated by an isopropyl β-d-1-thiogalactopyranoside-inducible lac promoter. FIG. 3B shows diverse read-throughs of RITs at different transcription initiation levels. FIG. 3C shows the plasmid design to measure the decay rate of mRNAs with different 3′-UTRs. FIG. 3D shows that the fluorescence intensity of GFP differed depending on the 3′-UTR. Error bars indicate the standard deviation of two biological replicate cultures. FIG. 3E shows the exponential decay of mRNA from pGFP. Error bars indicate the standard deviation of two biological replicates, each composed of technically triplicate qRT-PCR reactions. T_{1/2} indicates the mRNA half-life. FIG. 3F shows that the fluorescence intensity was directly correlated with the half-life of mRNA encoding GFP. FIG. 3G shows that computational modeling of fluorescence protein expression revealed a reliable prediction of the experimental measurements. Circles indicate experimental values from pGFP_empty and pGFP_rrnBT. Shades are ranges of the possible fluorescence intensity set by the standard error of the measured half-lives.

FIG. 4: Consistency and noise level of transcription terminator bioparts. FIG. 4A shows the read-through fraction of RITs in different experimental conditions. Scatter plots of the correlation between RIT read-throughs in diverse experimental conditions with M9 glucose medium. Error bars indicate the standard deviation of biological replicates. R2 indicates the Pearson's correlation coefficient. FIG. 4B shows the design of a genetic system expressing two fluorescence proteins using two independent promoters. FIG. 4C shows the results of flow cytometry measurements of fluorescence proteins expressed by two independent promoters with combinations of inducer concentrations. Each point represents a single cell. In the drawing, a indicates the slope of the linear regression line, and ηₑₓₜ and ηᵢₙₜ indicate the extrinsic and intrinsic noise components in RNA expression, respectively. FIG. 4D shows the results of flow cytometry measurement of the fluorescence proteins expressed by terminator bioparts. RT indicates the read-through of pDRA2 observed from overnight induced (with 10 UM IPTG) in cell cultures.

FIG. 5: Stable terminator bioparts can control metabolic pathways in a reliable and scalable manner. FIG. 5A shows the heterologous pathways for acetoin and 2,3-butanediol (2,3-BDO) biosynthesis from pyruvate. Abbreviations are as follows: alsS, acetolactate synthase; alsD, acetolactate decarboxylase; and bdh, butanediol dehydrogenase. FIG. 5B shows that the expression of bdh is dependent on the read-through of RITs located upstream. FIG. 5C shows the altered production of acetoin and 2,3-BDO in response to the RIT strengths. The titers of extracellular metabolites were measured after 24 h batch fermentation. Error bars indicate the standard deviations of the two biological replicate cultures, and pie charts indicate the relative expression of alsSD and bdh. FIG. 5D shows the metabolic flux valve system controlling the glycolytic flux for cellular resources and myo-inositol (MI) production. The expression of pfkA involves the metabolic valve of stable terminator bioparts. Abbreviations are as follows: zwf, glucose-6-phosphate dehydrogenase; pgi, glucose-6-phosphate isomerase; pfkA, 6-phosphofructokinase 1; and INO1, inositol-3-phosphate synthase. FIG. 5E shows the design of the flux-valved MI production pathway. The E. coli ΔpfkA Δzwf strain was used for maximum production and control efficiency. FIG. 5F shows the glucose consumption, MI production, and MI yield from the glucose (g/g) of E. coli harboring one of the pMI plasmids. The conv. sys. indicates the E. coli MG1655 wild-type (both pfkA and zwf were not perturbed) strain heterologously expressing INO1 without a metabolic valve. Error bars indicate the standard deviations of the two biological replicate cultures.

FIG. 6 shows the first-order decay model of pDRA2 transcripts. Panel (a) shows schematic representation of the two mRNA forms generated by pDRA2 system, and panel (b) shows the mathematical model prediction of pDRA2 compared to the measured fluorescence. Circles indicate individual experimental values, and lines indicate model prediction. Time variable is arbitrary unit.

FIG. 7 shows the quantification of noise in gene expression.

FIG. 8 shows the expression levels of the two fluorescence proteins by pDRA2 under different IPTG concentrations. Panel (a) shows that expression levels of GFP (green) and RFP (red) in the pDRA2_empty carrying E. coli had a linear correlation with the logarithm of IPTG concentration. R2 indicates the Pearson's correlation coefficient. Panel (b) shows that the expression of GFP (green) was saturated at IPTG concentration of 0.5 mM and the GFP intensity was linearly correlated with the logarithm of IPTG concentration before saturation. The expression of RFP (red) remained at a minimal level due to the strong termination of rrnB T1 terminator. Error bars indicate the s. d. of biological replicates.

FIG. 9 shows the correlation between structural parameters of T3PEs and read-through. Panel (a) shows a scatter plot depicting the folding energy of 70 bp upstream sequence of T3PEs to their most stable structure, panel (b) shows a scatter plot depicting the folding energy of stem loop structure at upstream of T3PEs, and panel (c) shows a scatter plot depicting the length of the stem in stem loop. R2 indicates the Pearson's correlation coefficient.

FIG. 10 shows the fold-change of read-throughs of RITs in diverse experimental conditions compared to M9 glucose medium.

FIG. 11 shows the transcript 5′-end mapping profiles near Group B terminators. The profiles of transcript 5′-mapping near Group B terminators T3PE-332 (a), T3PE-1238 (b), T3PE-447 (c), T3PE-936 (d), and T3PE-390 (e), which display the formation of stable transcript 5′-end. On the contrary, there is no stable 5′-end formation on Group A rrnB terminator (f).

FIG. 12 shows the yield of BDO conversion from acetoin with respect to the read-through of RITs. Error bars indicate the standard deviation of two biological replicate cultures. The line is a logarithmic regression of data points with a Pearson's correlation coefficient (R2) of 0.865.

FIG. 13 shows the growth curves of E. coli carrying MI production pathway, which are growth curves of E. coli MG1655 ΔpfkA Δzwf (a) and growth curves of E. coli eMS57 ΔpfkA Δzwf (b). Culture was induced with 0.1 mM IPTG initially. Error bars indicate the standard deviation of two biological replicates.

FIG. 14 shows a three-dimensional scatter plot of the relationship between terminator strength, growth rate, and MI production in MG1655 ΔpfkA Δzwf. Dashed lines are the orthogonal projection of each point on the XZ-plane.

### DETAILED DESCRIPTION OF THE INVENTION

Hereinafter, the present invention will be described in detail with reference to examples and experimental examples. However, these examples and experimental examples are given for specifically illustrating the present invention, and the scope of the present invention is not limited thereto.

### Materials and Methods

E. coli Strains, Media, and Culture

The E. coli strain K-12 MG1655, genome-reduced eMS57, and their derivatives were used in this study. The E. coli ΔpfkA Δzwf double-knockout strain was constructed using two sequential lambda recombination steps. Briefly, the zwf coding sequence was replaced with a kanamycin resistance cassette amplified from pKD13, using the pKD46 helper plasmid. The kanamycin resistance cassette was removed by flippase recombination, using the pCP20 plasmid. Then, pfkA was sequentially deleted using the same method. The double knockout strain was constructed, followed by pKD46 and pCP20 curing at a non-permissive temperature (42° C.). For the fluorescence reporter assay, overnight E. coli MG1655 culture, harboring the pDRA1, pDRA2, or pGFP plasmids, was inoculated into 400 μL of M9 glucose medium (47.75 mM Na2HPO4, 22.04 mM KH2PO4, 8.56 mM NaCl, 18.70 mM NH4CL, 2 mM MgSO4, 0.1 mM CaCl2, and 2 g/L glucose), with an initial optical density at 600 nm (OD 600 nm) of 0.05. Various concentrations of IPTG were added, as required. The orthogonality of transcript 3′-ends (T3PEs) was tested under M9 glycerol (2 g/L), M9 high-glucose (10 g/L), LB broth (BD Biosciences, San Jose, CA, USA), and TB (BD Biosciences). For the 2,3-butanediol (BDO) production experiment, E. coli MG1655 harboring pBDO was inoculated into 60 mL of M9 high-glucose medium containing 10 g/L glucose, 100 μg/mL ampicillin, and 0.1 mM IPTG, with an initial OD 600 nm of 0.05. The culture was grown aerobically in a 300 mL Erlenmeyer flask at 37° C. for 24 h in a rotary shaker. For the myo-inositol (MI) production experiment, the E. coli MG1655 or eMS57 ΔpfkA Δzwf double knockout strain harboring the pMI plasmid was inoculated into 60 mL of M9 high-glucose medium containing 10 g/L glucose, 100 μg/mL ampicillin, and 0.1 mM IPTG, with an initial OD 600 nm of 0.05. The culture was grown at 30° C., as previously described, and aerobically in a 300 mL Erlenmeyer flask for up to 48 h in a rotary shaker. Cell density (OD 600 nm) was monitored non-invasively using an OD-Monitor System (Taitec Corporation, Saitama, Japan) composed of an ODSensor-S and ODBox-A.

### Term-Seq

Term-Seq libraries were used as previous research results, with slight modifications. Briefly, RNA was extracted from E. coli MG1655 cultures sampled at the mid-log phase by using the RNAsnap™ method. Next, 5 μg of total RNA was treated with 2 U of RNase-free DNase I (NEB, Ipswich, MA, USA) for 15 min at 37° C. DNase I-treated RNA was purified using a phenol-chloroform-isoamyl alcohol extraction and ethanol precipitation. Ribosomal RNA (rRNA) was depleted from purified DNA-depleted RNA samples using the RiboZero rRNA Removal Kit for bacteria (Illumina, San Diego, CA, USA), according to the manufacturer's instructions. The RNA 3′ adaptor was ligated to 1 μg of rRNA-depleted RNA at 23° C. for 2.5 h, in 25 μL of a 3′ adaptor ligation reaction mixture: 1 μL of 150 μM RNA 3′ adaptor, 2.5 μL of 10×T4 RNA Ligase 1 Buffer (NEB), 25 U T4 RNA Ligase 1 (NEB), 2.5 μL of 10 mM ATP, 2 μL of dimethyl sulfoxide (DMSO), and 9.5 μL of 50% polyethylene glycol 8000 (PEG8000). The 3′-adaptor-ligated RNA was purified using Agencourt AMPure XP Beads (Beckman Coulter, Brea, CA, USA) and fragmented using the RNA Fragmentation Reagent (Ambion, Austin, TX, USA) at 72° C. for 90 s. The fragmented RNA was purified using Agencourt AMPure XP Beads and reverse transcribed using a SuperScript III First-Strand Synthesis System (Invitrogen, Carlsbad, CA, USA), according to the indications of the manufacturer, with 10 pmol RT primer. cDNA was purified using Agencourt AMPure XP beads, and the cDNA 3′ adaptor was ligated at 23° C. for 8 h in 25 μL of cDNA 3′ adaptor ligation reaction mixture: 1 μL of 150 μM cDNA 3′ adaptor, 2.5 μL of 10×T4 RNA Ligase 1 Buffer (NEB), 25 U T4 RNA Ligase 1 (NEB), 2.5 μL of 10 mM ATP, 2 μL of DMSO, and 9.5 μL of 50% PEG8000. Adaptor-ligated cDNA was purified using Agencourt AMPure XP beads. The final sequencing library was amplified and indexed using PCR amplification in 50 μL of a PCR reaction mixture: 1 U Phusion High-Fidelity DNA Polymerase (Thermo Fisher Scientific, Waltham, MA, USA), 10 μL of 5× Phusion HF Buffer (Thermo Fisher Scientific), 37.5 pmol of each primer (Amp_F and Amp_Index #_R), and 1 μL of 10 mM dNTP mix. The final sequencing library was amplified, and the PCR reaction was completed at a semi-plateau and included the following steps: initial activation at 98° C. for 30 s; 10 cycles of 98° C. for 30 s, 52° C. for 30 s, and 72° C. for 15 s; and final elongation at 72° C. for 30 s. The amplified sequencing libraries were subjected to two consecutive purifications using Agencourt AMPure XP beads. Sequencing was conducted for 50 cycles of a single-ended recipe using a HiSeq 2500 sequencer (Illumina).

### Term-Seq Data Processing and Transcript 3′-End Detection Using Machine-Learning

Term-Seq data were processed using the CLC Genomics Workbench (CLC Bio, Aarhus, Denmark). Raw reads were trimmed using the Trim Sequence Tool, with a quality limit of 0.05. Reads with two or more ambiguous nucleotides were discarded. Two random nucleotides located at the 5′- and 3′-ends that were attached during adaptor ligation were removed from the trimmed reads. The reads were mapped onto the MG1655 reference genome (NC_000913.3) with a mismatch cost of 2, indel cost of 3, length fraction of 0.9, and similarity fraction of 0.9. Mapping data were converted to the GFF file format. Briefly, the positions of 5′-ends of aligned reads that marked the RNA 3′-ends were extracted from the BAM file using Samtools and BEDTools. The 5′-ends in the genome were counted and written as a GFF file format using an in-house Python script. T3PEs were searched using an in-house Python script based on scikit-learn packages. To determine if a specific genomic position is a stable T3PE, the Term-Seq signals from −10 to +11 nt, relative to the position, were submitted to a machine classifier as a dataset, which provided a determination call for the position as its output. This was iterated throughout the genome for both strands to detect T3PE. The machine classifier was trained using a training set composed of 694 manually curated or RegulonDB termination sites, each comprising 191 positive and 503 negative calls. Two different machine classifiers, K-nearest neighbor (KNN) and a support vector machine, trained by the training set had a mean accuracy of 94.0% and 80.7%, respectively, upon cross-validation (trained with half of the randomly selected training set, with performance measured on the remaining half, iterated 1000 times). The KNN classifier was used for further termination site discovery. The machine was revised twice with a manual false call inspection. The Python script and KNN machine classifiers used in this study (pickled Python objects) are available in the GitHub repository (https://github.com/robinald/ML_Term-Seq).

### Transcript 5′-End Mapping Using Differential RNA-Seq

The 5′-end of the RNA was probed using dRNA-Seq. Briefly, rRNA-subtracted RNA was split into two samples. One sample was treated with 20 U RNA 5′ polyphosphatase (Epicentre, Madison, WI, USA) at 37° C. for 60 min. The other sample was treated with nuclease-free water. The dephosphorylated RNA adaptor was ligated to both samples using 5 U of T4 RNA ligase (Epicentre) at 37° C. for 90 min.

The adaptor-ligated RNA samples were purified, and cDNA was synthesized using the SuperScript III First-Strand Synthesis System with 3.125 pmol random nonamers. DNA libraries were amplified by PCR with Phusion High-Fidelity DNA Polymerase, using P5 and P7 index primers for 20 cycles. The amplified libraries were sequenced via the 50 cycles single-ended recipe on a HiSeq 2500 sequencer (Illumina). The amplified sequencing libraries were subjected to two consecutive purifications using Agencourt AMPure XP beads.

### RNA Structure Prediction and the Free Energy of Folding

RNA structure and the free energy of RNA folding were calculated from the 45 nt-long DNA sequence upstream of the T3PE using RNAfold software.

### Plasmid Construction.

The dual-reporter plasmid, pDRA1_empty, was constructed by inserting egfp into the mrfp1-expressing iGEM plasmid BBa_J04450-pSB1C3. Briefly, egfp DNA was amplified from the pTrc-egfp plasmid using the primers, egfp_F and egfp_R. The plasmid backbone was amplified using the pSB1C3_inv_F and pSB1C3_inv_R primers. The two DNA fragments were ligated using an In-Fusion Cloning Kit (TakaRa Bio, Shiga, Japan) according to the instructions of the manufacturer. The pDRA1_terminator plasmid was constructed by inserting terminator fragments into pDRA1_empty. Briefly, a 60-nt primer pair was annealed together in a 10 μL PCR reaction mixture: 0.2 U Phusion High-Fidelity DNA Polymerase (Thermo Fisher Scientific), 2 μL of 5× Phusion HF Buffer, 2 pmol of each primer (terminator_F and terminator_R), and 0.1 μL of 10 mM dNTP mix. The annealing reaction was performed at an initial activation temperature of 98° C. for 30 s, and then 12 cycles of 98° C. for 30 s, 52° C. for 30 s, 72° C. for 15 s, with a final elongation at 72° C. for 30 s. The annealed 100-bp dsDNA fragment was composed of a 15 nt sequence homologous to pDRA1_empty at each end and a 70 nt terminator fragment. All annealed products were inspected using gel electrophoresis and purified using a MinElute PCR Purification Kit (Qiagen, Hilden, Germany) according to the instructions of the manufacturer. The pDRA1_empty plasmid backbone was also PCR-amplified and linearized using the primers pSB1C3_inv_F and reporter_inv_R. The annealed terminator fragment (1.5 ng) and 10 ng of the pDRA1_empty plasmid backbone were incubated at 50° C. for 15 min in a 1 μL of a cloning reaction volume containing 0.2 μL of 5× In-Fusion HD Enzyme Premix. The pDRA2_terminator plasmid was constructed by inserting the egfp-terminator-mrfp1 DNA fragment, amplified with the transfer_assay_F and transfer_assay_R primers from the pDRA1_terminator plasmid, into the pTrcHis2A plasmid (Invitrogen) backbone, which was also PCR-amplified using the backbone_F and backbone_R primers. Cloning was performed using 10 and 8 ng of egfp-terminator-mrfp1 and pTrcHis2A plasmid backbones, respectively, in a 1 μL cloning reaction volume containing 0.2 μL of 5× In-Fusion HD Enzyme Premix. The pGFP_terminator plasmid was constructed by self-ligating the inverse PCR product that excluded the mrfp1 gene from the pDRA2_terminator plasmid, using the pGFP_F and pGFP_R primers. Self-ligation was performed in a 2.5 μL cloning reaction volume containing 0.5 μL of 5× In-Fusion HD Enzyme Premix and 20 ng inverse PCR product. The pDRA2_DualP plasmid was constructed by inserting the araC-araBAD promoter cassette into the pDRA2 plasmid by In-Fusion cloning. The araC-araBAD promoter cassette was PCR amplified from the pBADMyc-His C plasmid (Invitrogen) using the ara_F and ara_R primers. The pBDO_empty plasmid was constructed by the sequential insertion of chemically synthesized (IDT gBlock Gene Fragment) B. subtilis alsS-RBS-Aeromonas hydrophila alsD and codon-optimized Thermoanaerobacter brockii bdh into pTrcHis2A. First, the alsSD fragment was amplified using the alsS_pTrc_F and alsS_pTrc_R primers. The alsSD fragment was In-Fusion cloned into pTrcHis2A and amplified with the backbone_F and backbone_R primers. Then, bdh was PCR-amplified with the tbr_bdh_F and tbr_bdh_R primers, and In-Fusion cloned into PCR-amplified pTrcHis2A-alsSD using the alsSD_F and alsSD_R primers. The pBDO_terminator plasmid was constructed by restriction ligation of the terminator fragment amplified from the pDRA1_terminator plasmid, using the terminator_BDO_F and terminator_BDO_R primers, into pBDO_empty using Xhol and Spel sites.

The pMI_empty plasmid was constructed by cloning yeast INO1, E. coli pfkA, and the terminator fragment into pTrcHis2A. First, the INO1 structural gene was amplified from Saccharomyces cerevisiae CEN.PK genomic DNA, using the INO1_F and INO1_R primers. The INO1 gene fragment was In-Fusion cloned into the pTrcHis2A plasmid, which was amplified using the backbone_F and backbone_R primers. Next, pfkA was amplified from E. coli MG1655 genomic DNA using the pMI_pfkA_F and pMI_pfkA_R primers. The gene was In-Fusion cloned into the pTrcHis2A-INO1 plasmid amplified using the alsSD_F and pMI_INO1_R primers. The terminator fragment was cloned using restriction ligation with the Xhol and Spel sites in pMI_empty to generate the pMI_terminator.

### Fluorescence Measurement and Normalization

The fluorescence levels of the E. coli cultures were measured using a Synergy H1 Microplate Reader (BioTek Instruments, Winooski, VT, USA) 24 h after inoculation. For GFP, excitation with a 485 nm xenon flash and emission at 528 nm was measured with a gain of 90. For RFP, excitation with a 584 nm xenon flash and emission at 619 nm was measured with a gain of 120. Read-through values were calculated by dividing the RFP intensity by GFP and normalized by setting the read-through of pDRA1_empty as 1.

### Measurement of the Transcript Decay Rate

Cells were inoculated in a 250 mL Erlenmeyer flask containing 50 mL of M9 glucose medium supplemented with 100 μg/mL ampicillin and 1 mM IPTG. When the culture reached the exponential phase (OD 600 nm=0.3), 50 g/mL of rifampicin was added. Then, 10 mL of the rifampicin-treated culture was flash-frozen in liquid nitrogen at 0.5, 5, and 10 min after treatment. Each frozen culture was centrifuged at 4,000×g at 4° C. for 30 min. Total RNA was extracted from the cell pellets using the RNAsnap method. Next, 5 μg of total RNA was treated with 2 U RNase-free DNase I at 37° C. for 1 h. DNA-subtracted RNA was purified using phenol-chloroform extraction. Complementary DNA was synthesized from 500 ng of purified RNA using the SuperScript III First-Strand Synthesis System according to the indications of the manufacturer. Briefly, 500 ng of DNA-subtracted RNA, 25 ng of random hexamer, 0.5 μL of 10 mM dNTP mix, and nuclease-free water were mixed in 5 μL of a reaction volume. The mixture was incubated at 65° C. for 5 min and immediately placed on ice after incubation. Then, 5 μL of cDNA Synthesis Mix (containing 1 μL of 10× RT buffer, 2 μL of 25 mM MgCl2, 1 μL of 0.1 M dithiothreitol, 20 U RNaseOUT recombinant ribonuclease inhibitor, and 100 U SuperScript III reverse transcriptase) was added and incubated at 25° C. for 10 min. The mixture was incubated at 50° C. for 50 min, and then at 85° C. for 5 min. One unit of E. coli RNase H was incubated at 37° C. for 20 min to remove the RNA. Quantitative PCR was performed in 10 μL of a reaction volume containing: 5 μL of KAPA SYBR FAST qPCR Master Mix (Kapa Biosystems, Wilmington, MA, USA), 5 pmol of forward and reverse primers, 1 μL of 20× diluted cDNA, and 0.2 μL of 50×ROX High dye. The conditions of 40 PCR cycles were as follows: 95° C. for 10 s, 58° C. for 20 s, and 72° C. for 10 s. PCR was performed and monitored using a StepOnePlus Real-Time PCR System (Applied Biosystems, Foster City, CA, USA). When measuring RNA stability, 16S ribosomal RNA was assumed to be stable as its half-life was reported to exceed 10 min. The amount of egfp mRNA was measured using the −ΔΔCT method, with rrsA as an internal reference. All primers were designed using Primer-BLAST without non-specific binding to the E. coli MG1655 genome sequence (NC_000913.3).

The primer sequences used in the previous embodiments are shown in the following table.

TABLE 1

| Name | Sequence (5′ to 3′) |
|---|---|
| egfp_F | TCACACATACTAGAGAAAGAGGAGAAA |
| | TACTAGATGGTGAGCAAGGGCGAGGA |
| egfp_R | TCTTTGGATCCGAATTCTTACTTGTAC |
| | AGCTCGTCCA |
| pSB1C3_inv_F | ATTCGGATCCAAAGAGGAGAAATACTA |
| | GATGGCTT |
| pSB1C3_inv_R | CTCTAGTATGTGTGAAATTG |
| reporter_inv_R | TCTTACTTGTACAGCTCGTC |
| transfer_assay_F | TAAATAAGGAGGAATAAACCATGGTGA |
| | GCAAGGGCGAGGA |
| transfer_assay_R | TGATGATGATGGTCGACGGCTTAAGCA |
| | CCGGTGGAGTGAC |
| backbone_F | GCCGTCGACCATCATCATCA |
| backbone_R | GGTTTATTCCTCCTTATTTAATCGATA |
| | CAT |
| pGFP_F | CTAAATACATTCAAATATGT |
| pGFP_R | GAATGTATTTAGTCTCCTCTTTGGATC |
| | CGAAT |
| ara_F | ATTCGGATCCAAAGAGGAGAACGGTTC |
| | CTGGCCTTTTGCT |
| ara_R | ACGTCTTCGGAGGAAGCCATGGTTAAT |
| | TCCTCCTGTTAGCCC |
| alsS_pTrc_F | AAGGAGGAATAAACCATGTTGACAAAA |
| | GCAACAAAAGA |
| alsD_pTrc_R | ATGATGGTCGACGGCCTAACCCTCAGC |
| | CGC |
| tbr_bdh_F | CTCGAGGAATTCACTAGTAAAGAGGAG |
| | AAATACTAGATGGTGAAAGGCTTTGCG |
| | AT |
| tbr_bdh_R | TCCCTACTCTCGTTAAGCGAGAATGAC |
| | CACAG |
| alsSD_F | CGAGAGTAGGGAACTGCCAG |
| alsSD_R | GAATTCCTCGAGCTAACCCTCAGCCGC |
| | ACGGA |
| T3PE_BDO_F | TTAGCTCGAGGCTGTACAAGTAAGA |
| T3PE_BDO_R | CATCACTAGTTTCTCCTCTTTGGATCC |
| | GAAT |
| INO1_F | GAGGAATAAACCATGACAGAAGATAAT |
| | ATTGCTCCAA |
| INO1_R | ATGGTCGACGGCTTACAACAATCTCTC |
| | TTCGAATC |
| pMI_pfkA_F | CTCGAGGAATTCACTAGTAAAGAGGAG |
| | AAATACTAGATGATTAAGAAAATCGGT |
| | GTGT |
| pMI_pfkA_R | TCCCTACTCTCGTTAATACAGTTTTTT |
| | CGCGC |
| pMI_INO1_R | GAATTCCTCGAGTTACAACAATCTCTC |
| | TTCGAATC |
| RNA 3′ adaptor | p-NNAGATCGGAAGAGCGTCGTGT- |
| | AmMO |
| RT primer | TCTACACTCTTTCCCTACACGACGCTC |
| | TTC |
| cDNA 3′ | p-NNAGATCGGAAGAGCACACGTCTGA |
| adaptor | ACTCCAGTCAC-AmMO |
| Amp_F | AATGATACGGCGACCACCGAGATCTAC |
| | ACTCTTTCCCTACACGACGCTCT |
| Amp_Index#_R | CAAGCAGAAGACGGCATACGAGATNNN |
| | NNN(Index)GTGACTGGAGTTCAGAC |
| pfkA_KO_F | AGACTTCCGGCAACAGATTTCATTTTG |
| | CATTCCAAAGTTCAGAGGTAGTCGTGT |
| | AGGCTGGAGCTGCTTC |
| pfkA_KO_R | GCTTCTGTCATCGGTTTCAGGGTAAAG |
| | GAATCTGCCTTTTTCCGAAATCACTGT |
| | CAAACATGAGAATTAA |
| pfkA_KO_confirm_F | GTTGGATCACTTCGATGTGC |
| pfkA_KO_confirm_R | TGACAAAGTGCGCTTTGTCC |
| zwf_KO_F | CAAGTATACCCTGGCTTAAGTACCGGG |
| | TTAGTTAACTTAAGGAGAATGACGTGT |
| | AGGCTGGAGCTGCTTC |
| zwf_KO_R | GCGCAAGATCATGTTACCGGTAAAATA |
| | ACCATAAAGGATAAGCGCAGATACTGT |
| | CAAACATGAGAATTAA |
| zwf_KO_confirm_F | ACAGTGCACCGTAAGAAAAT |
| zwf_KO_confirm_R | ATCCGCACGAGGCCTGAAAG |
| RNA adaptor | ACACUCUUUCCCUACACGACGCUCUUC |
| for transcript 5′ | CGAUCU |
| end mapping | |
| Random | CCTGCTGAACCGCTCTTCCGATCTNNN |
| nonamer | NNNNNN |
| P5 primer | AATGATACGGCGACCACCGAGATCTAC |
| | ACTCTTTCCCTACACGACGCTCTTCCG |
| | ATCT |
| P7 primer | CAAGCAGAAGACGGCATACGAGAT- |
| | [6 nt index]-GTGACTGGAGTT |
| | CAGACGTGTGCTCTTCCGATCT |

Mathematical Modeling of the mRNA Levels

The change in GFP mRNA concentration per unit time in an E. coli population carrying the pGFP plasmid was expressed in a first-order decay model, as shown in Equation 1:

$$\frac{d [ {mRNA}_{GFP} ] ( t )}{dt} = {k}_{TRX} - {k}_{decay} [ mRNA ] ( t ) \qquad \{ Equation 1 ]$$

where [mRNA_{GFP}](t) is the GFP mRNA concentration, K_{TRX} is the constant transcription rate, and k_{decay} is the mRNA decay rate. Thus, the mRNA concentration is expressed by Equation 2, by solving the first-order differential equation of Equation 1, with an initial value of [mRNA]|ₜ₌₀=0.

$$[ mRNA ] ( t ) = \frac{{k}_{TRX}}{{k}_{decay}} ( 1 - {e}^{- {k}_{decay} t} ) \qquad [ Equation 2 ]$$

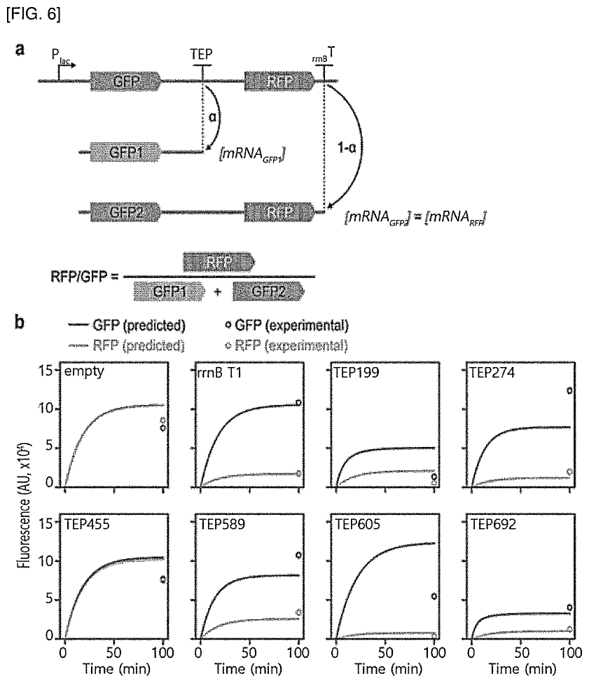

GFP protein synthesis and fluorescence were assumed to be linearly proportional to the mRNA level. In the simulation of pDRA2, a model was built in a similar manner, considering transcription termination (Equation 3 and FIG. 6). The model was based on two mRNA species. One is mRNA terminated by the terminator, which encodes only GFP:

$$\frac{d [ {mRNA}_{GFP 1} ] ( t )}{dt} = α {k}_{TRX} - {k}_{d ; terminator} \times [ {mRNA}_{GFP 1} ] ( t ) , \qquad [ Equation 3 ]$$

where [mRNA_{GFP1}](t) is the concentration of mRNA terminated by the terminator; K_{d;TEP} is the decay rate of the mRNA with a terminator on its 3′-UTR; abd α is a transcription termination strength of 0 for 100% read-through and 1 for 100% transcription termination.

The other mRNA is transcribed by read-through and encodes both GFP and RFP (Equations 4 and 5).

$$\frac{d [ {mRNA}_{GFP 2} ] ( t )}{dt} = ( 1 - α ) {k}_{TRX} - {k}_{d ; rrn BT} \times [ {mRNA}_{GFP 2} ] ( t ) \qquad [ Equation 4 ]$$

$$\frac{d [ {mRNA}_{RFP} ] ( t )}{dt} = ( 1 - α ) {k}_{TRX} - {k}_{d ; rrn BT} \times [ {mRNA}_{RFP} ] ( t ) , \qquad [ Equation 5 ]$$

where [mRNA_{GFP2}] (t) and [mRNA_{RFP}] (t) are the read-through mRNA concentrations, and K_{d;rmBT} is the decay rate of the mRNA with the terminator at the end. Thus, the collective concentration of GFP-expressing mRNA was calculated using Equation 6, and the RFP-expressing mRNA was expressed by Equation 7.

$$[ {mRNA}_{GFP} ] = \frac{α {k}_{TRX}}{{k}_{d ; TEP}} ( 1 - {e}^{- {k}_{d ; TEP} t} ) + \frac{( 1 - α ) {k}_{TRX}}{{k}_{d ; rrh BT}} \times ( 1 - {e}^{- {k}_{d ; rrh BT} t} ) \qquad [ Equation 6 ]$$

$$[ {mRNA}_{RFP} ] = \frac{( 1 - α ) {k}_{TRX}}{{k}_{d ; rrh BT}} \times ( 1 - {e}^{- {k}_{d ; rrh BT} t} ) \qquad [ Equation 7 ]$$

The read-through (RFP/GFP) of the saturated culture can be expressed by dividing the saturated (t→∞) RFP concentration by GFP (Equation 8).

$$\frac{RFP}{GFP} = \frac{( 1 - α ) {k}_{d ; TEP}}{α {k}_{d ; rrh BT} + ( 1 - α ) {k}_{d ; TEP}} \qquad [ Equation 8 ]$$

Finally, the termination strength a can be calculated using Equation 8, with the measured parameters (Equation 9).

$$α = \frac{{k}_{d ; TEP} ( 1 - ( \frac{RFP}{GFP} ) )}{( {k}_{d ; rrh BT} - {k}_{d ; TEP} ) ( \frac{RFP}{GFP} ) + {k}_{d ; TEP}} \qquad [ Equation 9 ]$$

### Flow Cytometry

Cells harboring pDRA2 or pDRA2_DualP were aerobically cultured in M9 glucose medium for 24 h at 37° C. with IPTG (1 or 10 μM) and L-arabinose (1, 10, or 30 mM). The cell culture was diluted 10-fold in PBS and analyzed on an S3e Cell Sorter (Bio-Rad, Hercules, CA, USA). Approximately 100,000 events were collected and analyzed.

### Noise Definition and Data Parameterization

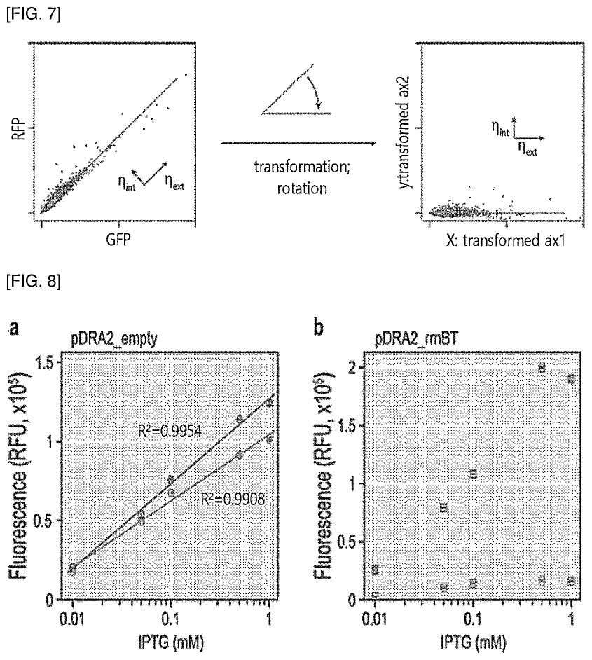

The GFP and RFP intensities of the entire population were plotted, and a linear regression line was obtained. Noise was defined as the coefficient of variation (CV; the standard deviation divided by the mean) as in previous research. Noise can be further decomposed into extrinsic and intrinsic noise that are independent and orthogonal to each other To calculate the two orthogonal noise components, the scatter plot showing the flow cytometry data was rotated by the slope of the linear regression line (FIG. 7). The CV of the extrinsic (along the horizontal axis) and intrinsic (along the vertical axis) components were then calculated.

### Quantification of Acetoin, BDO, and MI

One milliliter of a 24 h E. coli pBDO culture or a 48-h E. coli pMI culture was collected in 1.5 mL of microcentrifuge tubes and centrifuged at 16,000×g for 1 min. Supernatants were filtered using a syringe filter (0.2-μm pore size). The filtrate (200 μL) was transferred to a 2 mL high-performance liquid chromatography (HPLC) vial containing a 250 μL glass vial insert. Then, 20 μL of each sample was analyzed using HPLC with a system comprising a model 2414 refractive index detector (Waters Corporation, Milford, MA, USA), a 1525 binary HPLC pump (Waters), a Metacarb 87H (7.8×300 mm) HPLC column (Agilent Technologies, Santa Clara, CA, USA), and a 2707 Autosampler (Waters). The mobile phase was 0.007 N sulfuric acid, the flow rate was 0.6 mL/min, and the oven and detector temperatures were 50° C.

### Statistical Analysis

Term-Seq was conducted with three biological replicates, and all 1,629 T3PEs were supported by two or more replicates. Read-through value distributions according to the T3PE categories were compared using a two-sided t-test for unequal variance (Welch's t-test). All statistical analyses were performed using the SciPy package. Comparisons with a p-value<0.01 were considered significant in this study.

### Results and Discussion

### Identification of Multiple 3′ Transcript End Classes

The transcript 3′-ends (T3PE) classes in the E. coli MG1655 transcriptome were attempted to be identified using Term-Seq. The machine-learning analysis was used to detect 1,583, 1,795, and 1,951 T3PEs from each biological replicate. Among these, 1,629 T3PEs observed from two or more biological replicates were stable T3PEs.

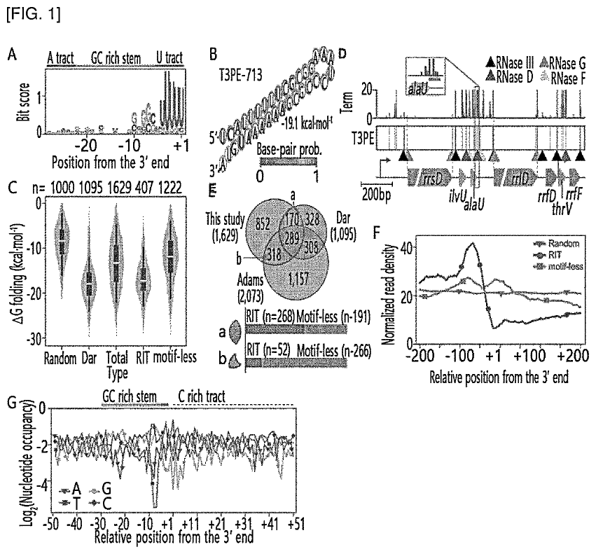

As a conserved sequence motif of rho-independent terminators (RIT), 407 T3PEs were identified (FIG. 1A). The sequences upstream of these T3PEs contained conserved GC-rich sequences that form the stem of a hairpin structure and a consecutive U-stretch (FIG. 1B), characteristic of intrinsic terminators. The RITs had a strong folding free energy compared to random genomic positions (FIG. 1C).

The 1,222 T3PEs without a conserved sequence motif (motif-less T3PEs) were unlikely to form a stem-loop structure because of their relatively high ΔG_{folding}. Among them, 413 motif-less T3PEs were located on the ribosomal RNA (rRNA)-transfer RNA (RNA) operons, which matched the rRNA-tRNA processing sites (FIG. 1D). The processive nuclease activity of RNase D was also observed (FIG. 1D, inset), indicating nucleotide-level accuracy of the Term-Seq results. The remaining 809 motif-less T3PEs may represent non-canonical types of transcriptional terminators, or stable transcript ends that are independent of termination, such as intermediates of RNA processing. However, these T3PEs are unlikely to be footprints of RNA degradation during the experimental procedure because they were observed in two or more biological replicates.

To cross-validate the classification, 1,629 T3PEs were compared with previously reported T3PEs in E. coli BW25113 or MG1655 (Dar, D. and Sorek, R. (2018) High-resolution RNA 3-ends mapping of bacterial Rho-dependent transcripts. Nucleic Acids Res., 46, 6797-6805; Adams, P. P., Baniulyte, G., Esnault, C., Chegireddy, K., Singh, N., Monge, M., Dale, R. K., Storz, G. and Wade, J. T. (2021) Regulatory roles of Escherichia coli 5-UTR and ORF-internal RNAs detected by 3-end mapping. Elife, 10, e62438). The 1,095 T3PEs in E. coli BW25113 were detected with positional constraints (within 150 bp downstream of genes), while the other 2,073 T3PEs in E. coli MG1655 were reported without any constraints.

With 3 nt accuracy, 459 T3PEs were found in both E. coli MG1655 and BW25113 (FIG. 1E). These shared T3PEs were mostly located in the intergenic region, and more than half were RITs (n=268, 58.4%). Conversely, motif-less T3PEs (n=1,031, 88.1%) formed a large proportion of the remaining 1,170 T3PEs detected in our study. Considering the tendency of detecting transcriptional terminators due to the positional constraint in the previous report, T3PEs that were not transcriptional terminators could be determined through the detection method of this study. Another report (Adams, P. P., Baniulyte, G., Esnault, C., Chegireddy, K., Singh, N., Monge, M., Dale, R. K., Storz, G. and Wade, J. T. (2021) Regulatory roles of Escherichia coli 5′ UTR and ORF-internal RNAs detected by 3′ end mapping. Elife, 10, e62438) on E. coli MG1655 showed greater agreement with T3PE detected in this study (FIG. 1E).

Of the 1,170 T3PEs that did not overlap with E. coli BW25113, 318 T3PEs agreed with previously detected motif-less T3PEs (n=273, 85.5%), which were consistently observed in multiple experiments.

Finally, 852 novel T3PEs composed of 87 RITs and 765 motif-less T3PEs were identified. To assess the functions of the various T3PEs, including motif-less T3PEs, the RNA-Seq profiles in close vicinity were investigated. A meta-analysis of 100,000 random genome positions presented no fluctuation in transcript signals within the 400 nt sequence window (FIG. 1F). The RNA-Seq signal was clearly depleted after RITs, indicating strong transcriptional termination. In contrast, the RNA-Seq profile of neighboring motif-less T3PEs displayed no significant transcript depletion and formed a stable profile.

A few motif-less T3PE (n=41) matched with previously reported Rho-dependent terminators. Although these had sequence features of Rho-dependent terminators (FIG. 1G), a weakly-conserved Rho-dependent terminator motif was not discovered in this case. Thus, the majority of motif-less T3PEs are related to biological processes other than transcriptional termination.

### Determination of Transcription Termination Strength

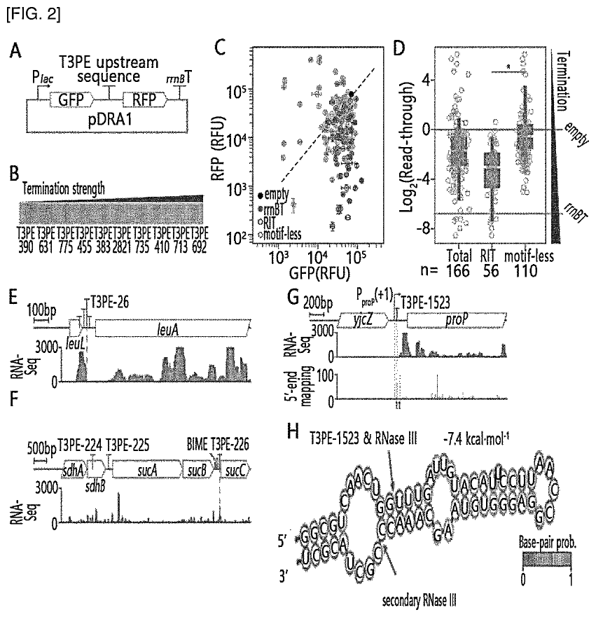

To further investigate the identified T3PEs, a dual reporter assay system (pDRA1) containing a 70 nt DNA fragment obtained upstream of a T3PE was devised (FIG. 2A). The pDRA1 plasmids containing one of the 50 randomly selected RITs and 100 motif-less T3PEs (excluding T3PEs located in the rRNA operons) were constructed. Different T3PEs led to different expression levels of fluorescent proteins located downstream of T3PE (FIG. 2B). A fraction of the two different fluorescence signals was used to quantitatively measure the terminator strengths, hereafter referred to as “read-through fraction”. The strong rrnB T1 terminator (rrnBT) displayed a read-through fraction of 0.009, indicating a termination of 99.1% of the transcription (FIG. 2C). The RITs located downstream of tRNA, asnV (T3PE-605) and leuW (T3PE-211), mediated termination as effectively as rrnBT. The read-through fractions from E. coli cultures carrying one of the 150 T3PE-carrying pDRA1 plasmids ranged from 0.004 to 91.4. The RITs displayed an average read-through fraction of 0.294 (FIG. 2D). Conversely, motif-less T3PEs showed significantly weaker transcription termination than RITs (FIG. 2D). Further analyses indicated that the 545 motif-less T3PEs could be the products of biological processes other than transcriptional termination. For example, T3PE-26 was located downstream of a leuABCD operon leader peptide, LeuL (FIG. 2E). LeuL is responsible for the post-transcriptional attenuation of leucine biosynthesis in response to excess leucine. Furthermore, the location of T3PE-226 coincides with that of the bacterial interspersed mosaic element (BIME), a palindromic repeat sequence throughout the E. coli genome (FIG. 2F). BIMEs are occasionally located in the middle of polycistronic mRNAs and play a role in differential gene expression that is co-transcribed as a single mRNA by stabilizing upstream genes. T3PE-1523 perfectly matched a well-known RNase III digestion site on the 5′-UTR of the proline transporter (ProP) mRNA, which modulates ProP expression in response to hyperosmotic stress (FIGS. 2G and 2H). RNA digestion resulted in the generation of 5′- and 3′-ends at the cleavage site. The transcript 5′-end mapping was performed using dRNA-Seq to assess the other cleavage end. A stable transcript 5′-end generated by RNase III processing was also observed in transcript 5′-end mapping (FIG. 2G). The 5′-end was not generated by transcription initiation on the promoter, as the transcription start site can be distinguished by differential RNA-Seq. T3PE-1523 cleavage on GFP-RFP mRNA in pDRA1 led to a marked increase (54.0 times) in RFP intensity compared to GFP. This is possibly due to the exposure of the ribosome binding site of RFP and the nucleolytic degradation of GFP mRNA at its 3′-end after RNase III cleavage.

The experimental results identified that motif-less T3PEs are independent of transcriptional termination. Using the pDRA1 assay, the capability of Term-Seq to detect multiple E. coli T3PE classes produced by transcriptome reshaping, RNA processing, cis-regulation, and transcriptional terminators was investigated.

### The Relationship Between Termination and Expression Level

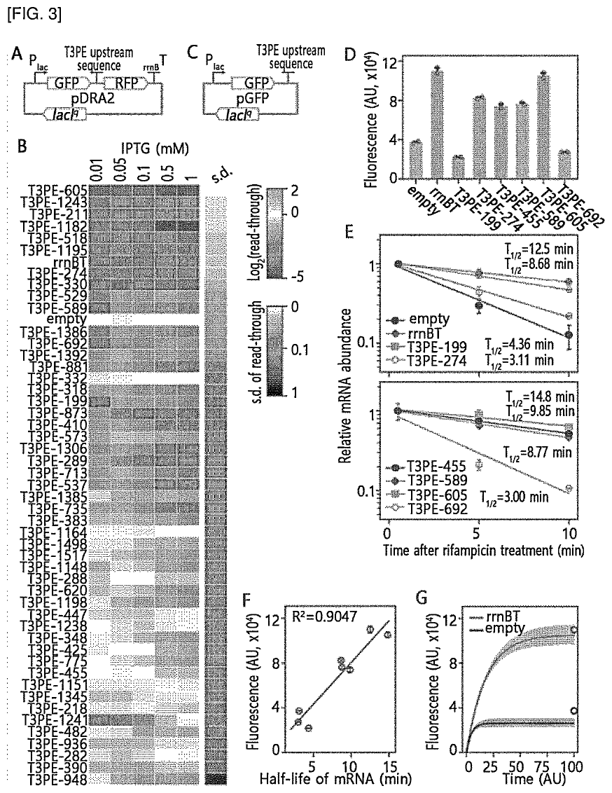

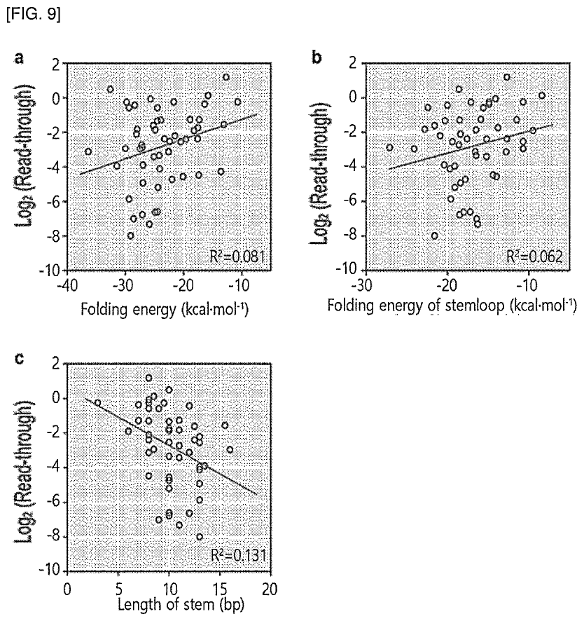

As multiple RITs exhibit a wide range of transcription termination strengths, the terminators at different gene expression levels were examined to assess whether the termination strength remained the same. Two control sequences (empty and rrnBT) and 49 RITs were tested under the control of the inducible Lac promoter (pDRA2, FIG. 3A). The fluorescence level increased logarithmically at different isopropyl 3-D-1-thiogalactopyranoside (IPTG) concentrations (FIG. 8). Strikingly, the termination levels of many RITs varied according to the transcription level (FIG. 3B). The transcription termination was generally stronger at higher expression levels, likely because of local resource depletion, such as NTPs, which lowers the elongation rate so that RNA polymerase can easily dissociate from its template. This variation may explain why precise termination strength prediction is difficult, thereby limiting the standardization of terminator bioparts (FIG. 9). It was also found that the termination strength reported by the two fluorescent proteins may be different from the actual transcriptional termination event. A thorough inspection of the data revealed considerable variations in GFP intensity. For example, the GFP intensity of pDRA1_rrnBT was 127,583 arbitrary units (AU), whereas the GFP intensity of pDRA1_empty was only 70,548 AU (FIG. 2C). It was hypothesized that the T3PE sequence at the 3′-end of the transcript is involved in transcript stability. In such cases, the differential degradation of the terminated gfp-only and read-through gfp-rfp transcripts causes the ratio of the two fluorescence signals to differ from the initial transcript ratio set by termination.

To test this hypothesis, rfp was removed from the pDRA2 plasmid to construct a pGFP plasmid (FIG. 3C). Since transcripts generated by different pGFP plasmids have the same 5′-UTR, coding sequence, and plasmid backbone, the difference in GFP intensity should originate solely from the T3PE sequence located in the 3′-UTR. The pGFP assay results indicated that E. coli harboring different pGFP plasmids displayed a maximum 5.0-fold difference in the fluorescence intensity (FIG. 3D). The time-course measurement of mRNA abundance at 0.5, 5, and 10 min after transcription inhibition through treatment with the transcription inhibitor, rifampicin, revealed an exponential cellular mRNA decay (FIG. 3E). The mRNA half-life varied from 3.0 to 14.8 min, consistent with the magnitude of GFP intensity variance. Moreover, the GFP intensity was directly proportional to the mRNA half-life (FIG. 3F).

Then, a first-order decay model of the GFP transcript was constructed to determine whether differential mRNA decay resulted in a difference in fluorescence. Computational modeling confirmed that the measured decay rate of the mRNAs was sufficient to induce a large difference in the GFP intensity of pGFP (FIG. 3G). Next, a mathematical pDRA2 model was constructed using the same decay model to examine whether the differential decay of gfp-only and read-through transcripts resulted in a significant discrepancy between the measured read-through and termination strengths (FIG. 6). Regarding pDRA2_rrnBT, where the terminators on gfp and rfp mRNA were the same, the strengths of the measured termination and modeled termination were identical. However, the termination strength calculated from the model, which considered the mRNA decay rates, differed significantly from the strength measured using the two fluorescence signals.

TABLE 2

| | Read- | 1-(read- | α (termination | % |
|---|---|---|---|---|
| T3PE | through | through) | strength) | deviation |
| rrnBT | 0.162 | 0.838 | 0.838 | 0.00% |
| T3PE-199 | 0.416 | 0.584 | 0.801 | 27.09% |
| T3PE-274 | 0.158 | 0.842 | 0.885 | 4.86% |
| T3PE-455 | 0.976 | 0.024 | 0.030 | 20.00% |
| T3PE-589 | 0.315 | 0.685 | 0.674 | 1.63% |
| T3PE-605 | 0.063 | 0.937 | 0.926 | 1.19% |
| T3PE-692 | 0.305 | 0.695 | 0.905 | 23.20% |

For example, T3PE-692 showed a moderate level of read-through with a strength of 0.31 in the pDRA2 assay but was considered a strong terminator based on the model-calculated termination strength of 0.91. This example highlights the difficulties in characterizing terminators, and this discrepancy is based on one of the complex processes governing transcriptional termination. These processes include the binding strength of RNA polymerase to template DNA, RNA decay, the energy required to denature dsDNA to maintain the transcription bubble, poly-U-stretch, partial rho-factor overlap, and secondary stem-loop structures.

### Consistency and Noise Levels of Transcription Terminator Bioparts

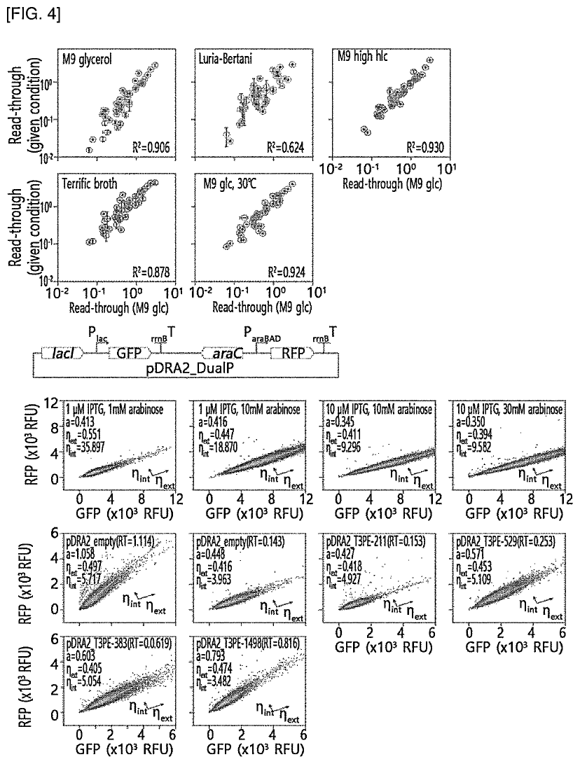

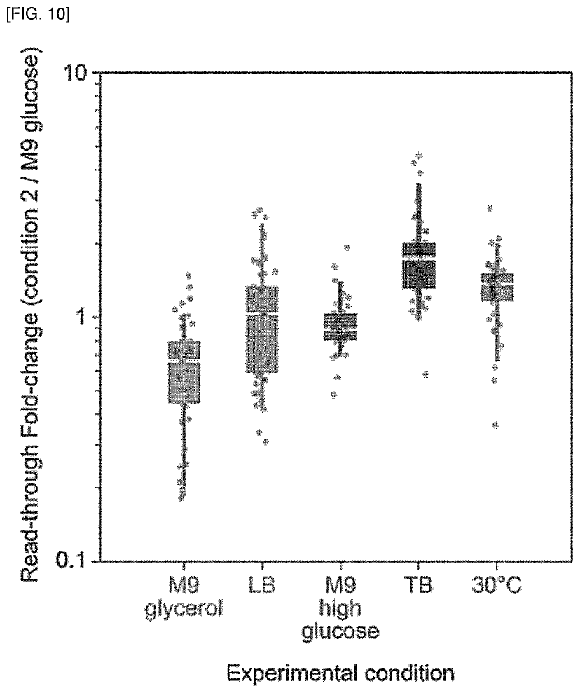

Next, it was examined whether the termination strengths of RIT were reliable under different experimental conditions. In addition to the M9 glucose medium previously tested, an additional incubation temperature (30° C.) and four different culture media, M9 glycerol, M9 high glucose (10 g/L), Luria-Bertani (LB), and Terrific Broth (TB), were tested. As RITs are independent of trans-acting elements, changes in transcription termination under various experimental conditions were negligible. As expected, the read-through fractions of RITs under the five additional conditions strongly correlated with the M9 glucose (2 g/L) medium. Specifically, read-through fractions of the tested RITs in high glucose (10 g/L) M9 medium were almost identical to those in normal M9 glucose, with a Pearson's R2 correlation of 0.93 and an average fold-change of 0.96 (FIG. 4A). Similarly, RITs had an average fold-change of 1.09 in LB medium. Unlike the high-glucose M9 and LB media, read-through fractions of RITs grown in M9 glycerol, TB media, and at 30° C. deviated from those grown in M9 glucose medium with mean fold-changes of 0.66, 1.84, and 1.34, respectively (FIG. 10). These deviations reflect different growth rates and energy levels, which may change the transcription kinetics, such as elongation rates, and could result in a transcriptome-wide termination shift.

The transcription elongation rate of E. coli depends on the growth rate, as evidenced by the slower elongation rate of E. coli grown in glycerol minimal medium compared to that grown in minimal glucose medium. The slower elongation rate in M9 glycerol medium increases susceptibility of RNA polymerase to a transcriptional pause at the terminator, resulting in stronger termination. In contrast, growth in TB resulted in a high elongation rate and reduced transcription termination. Cells grown in M9 glucose medium at 30° C. had a 1.34-fold increase in read-through, compared to cells grown at 37° C. The pDRA2 assay results obtained under several experimental conditions indicate that rho-independent transcription terminators in E. coli present consistent relative strength. However, the overall absolute strength of the entire termination shifts according to the global cellular changes in transcription kinetics and trans-acting elements, such as the elongation rate and possibly the alarmone level. This complex termination behavior must be considered in synthetic genetic circuitry. Furthermore, the experimental fluctuations of a genetic system composed of terminator bioparts were compared with those of conventional systems. Previous assessments of stochastic gene expression revealed two independent noise components (defined by the coefficient of variation, which is the standard deviation divided by the mean) that contribute to the overall fluctuation of cellular behavior. Extrinsic noise (ηₑₓₜ) is induced by different states, such as transcriptional/translational machinery concentrations and cellular energy states that cause variation in overall protein expression. The other component, intrinsic noise (ηᵢₙₜ), is caused by molecular events, such as random binding of RNA polymerases, ribosomes, and transcription factors, even when cells are assumed to have the same cellular state. Thus, the present study constructed an expression system that expressed two different fluorescent proteins from two different promoters to set a reference (FIG. 4B). Cells expressing GFP and RFP by the lac promoter and araBAD promoter had ηₑₓₜ and ηᵢₙₜ values of 0.451 and 18.4, respectively, yielding a total noise (ηₜₒₜ) of 18.4 (FIG. 4C). When terminator bioparts were used, ηᵢₙₜ was 4.08-fold lower than that of the dual promoter system (mean of 4.53) (FIG. 4D), whereas next remained the same (1.04-fold increase). The noise assessment indicated a 4.07-fold lower total system noise by reducing the sources of biological variation in transcription initiation (e.g. RNAP binding) and transcription factor regulation. This highlights a significant advantage of terminator bioparts over conventional regulatory systems that use multiple promoters and ribosome-binding sites.

### Development of Metabolic Flux Valves Based on Terminator Bioparts

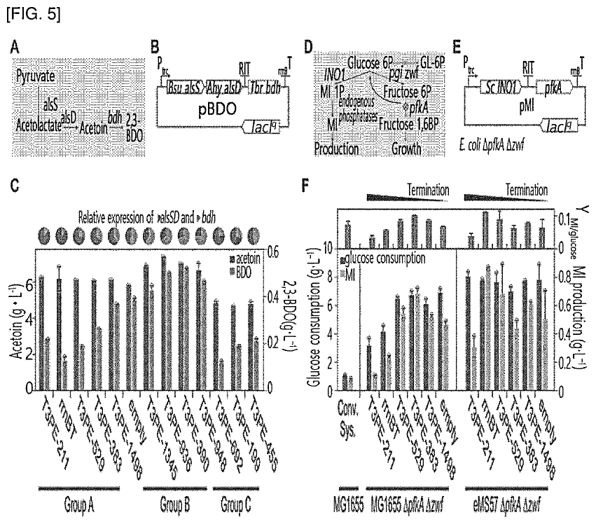

Various elements are used to regulate the expression of multiple genes in synthetic biology. As genetic circuits become more complicated to accommodate multiple connections and regulations, more bioparts are required. Thus, it was examined whether RITs with different termination strengths can function as reliable regulatory bioparts to provide additional means of regulating synthetic pathways with fewer bioparts. Acetoin and 2,3-butanediol (2,3-BDO) were selected as target products. Acetoin is produced by several bacteria to store the carbon flux overflow for later use and prevent the accumulation of acidic cellular metabolites. Acetolactate (AlsS) and acetolactate decarboxylase (AlsD) catalyze the condensation of two pyruvate molecules into a single acetoin molecule (FIG. 5A). Additionally, 2,3-BDO is an important organic molecule used to produce synthetic rubber. Butanediol dehydrogenase (Bdh) catalyzes the one-step conversion of acetoin to 2,3-BDO (FIG. 5A).

Three RIT groups were selected to control the system expression. The first group, Group A, contained five RITs (T3PE-211, rrnBT, 383, 529, and 1498), which had read-throughs of <1 and relatively stable read-through fractions at different transcription rates (FIG. 3B). The RITs in Group B (T3PE-1345, 936, 390, and 948) had read-through values>1. Therefore, downstream gene expression was higher than the upstream genes. The last group, Group C, was composed of three RITs that shortened the mRNA half-life when located at the 3′-UTR of an mRNA, as shown in the pGFP assay. These were located downstream of alsSD in the polycistronic 2,3-BDO synthetic plasmid pBDO (FIG. 5B). Bdh expression was controlled by the RIT read-through fraction, which determined the relative flux between acetoin and 2,3-BDO from pyruvate.

Then, 24 h after the addition of IPTG (0.1 mM), E. coli strains harboring one of the pBDO plasmids with RITs from Group A showed various 2,3-BDO titers due to various expression levels of bdh by different terminator read-through fractions (FIG. 5C). The amount of 2,3-BDO produced by pBDO_rrnBT, harboring the rrnB T1 terminator upstream of bdh, was 0.126 g/L. The BDO titer increased as the termination strength decreased, such that the fully induced negative control cells (pBDO_empty) produced 0.382 g/L of 2,3-BDO, the maximum titer of Group A RITs. More importantly, Group B RITs could support greater 2,3-BDO production, even greater than pBDO_empty, which has no transcription termination activity. In theory, bdh expression should always be lower than that of the upstream genes since the transcriptional terminator is located upstream of bdh. However, a higher 2,3-BDO production with the Group B RITs (maximum 0.513 g/L) was observed. This was consistent with the pDRA2 assay of the Group B RITs with read-through values>1. This phenomenon was not due to a promoter sequence that might overlap with T3PE. However, RFP expression was not observed in the pDRA2 assays of Group B RITs when they were not induced (Table 3).

TABLE 3
Fluorescence intensity of saturated culture of E. coli
containing pDRA2 plasmid when not supplemented with IPTG

| | | GFP | GFP | RFP | RFP | Read- |
|---|---|---|---|---|---|---|
| T3PE | Category | AVG | SD | AVG | SD | through |
| empty | Negative | 18333.8 | 282.5 | 18333.8 | 411.2 | 1.000 |
| | control | | | | | |
| rrnBT | Positive | 18810.5 | 98.3 | 1238.0 | 63.1 | 0.066 |
| | control | | | | | |
| T3PE-1345 | RIT | 25592.3 | 485.6 | 38852.5 | 309.7 | 1.518 |
| T3PE-936 | RIT | 21551.6 | 705.9 | 11414.8 | 4.7 | 0.530 |
| T3PE-390 | RIT | 15621.9 | 15.6 | 27377.4 | 527.5 | 1.752 |
| T3PE-948 | RIT | 14546.8 | 327.4 | 15080.1 | 108.2 | 1.037 |

(RFP expression indicates innate promoter activity of 70 bp T3PE sequence. AVG and SD indicate average and standard deviation of two replicate cultures.)

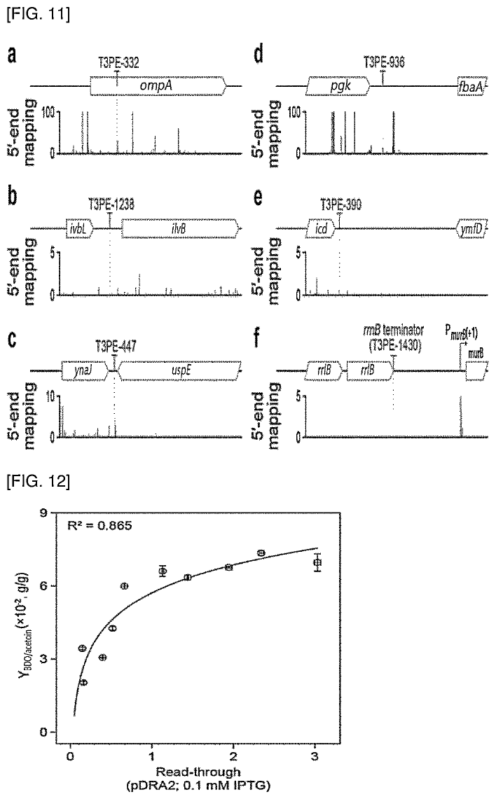

The behavior of Group B terminators, which increases the expression of downstream genes compared to that of upstream genes, was counter intuitive. Thus, transcript 5′-end mapping, which captures the stable transcript 5′-end in a cell, was performed. Among the 13 Group B terminators examined by the pDRA2 assay, five had stable transcript 5′-ends that were not produced by transcription initiation at their close vicinity, indicating active post-transcriptional RNA processing near the terminator (FIG. 11). Thus, increased ribosome entry and RBS availability induced by post-transcriptional cleavage, generates a new 5′-UTR, which may be responsible for the high expression of downstream genes. Regardless of the reason, the 2,3-BDO production continuously increased as the read-through fraction increased. The production reached a plateau at a read-through of 2.343 (T3PE-390). Saturation of the 2,3-BDO titer at the high read-through fraction was possibly due to the saturation of the cellular resource to convert acetoin to 2,3-BDO, that is, NADH. This illustrates the ability of T3PE bioparts to support a wide range of transcription ratios among genes. Since read-through values can exceed 1, the system requires no additional modification to change the gene order to accomplish higher downstream gene expression. Overall, as the read-through of T3PE increased, a greater metabolic flux was directed toward 2,3-BDO from acetoin. The 2,3-BDO to acetoin ratio was logarithmically proportional to the read-through strength of the T3PEs with high correlation, indicating the reliability of T3PEs as synthetic bioparts for modifying metabolic flux (FIG. 12). Finally, Group C RITs that produced short-lived mRNAs were tested. As expected, E. coli harboring pBDO with Group C RITs produced less acetoin and 2,3-BDO than cells with pBDO consisting of RITs in other groups (FIG. 5C). This highlights the importance of selecting an appropriate terminator biopart to achieve the desired functionality of a genetic circuit.

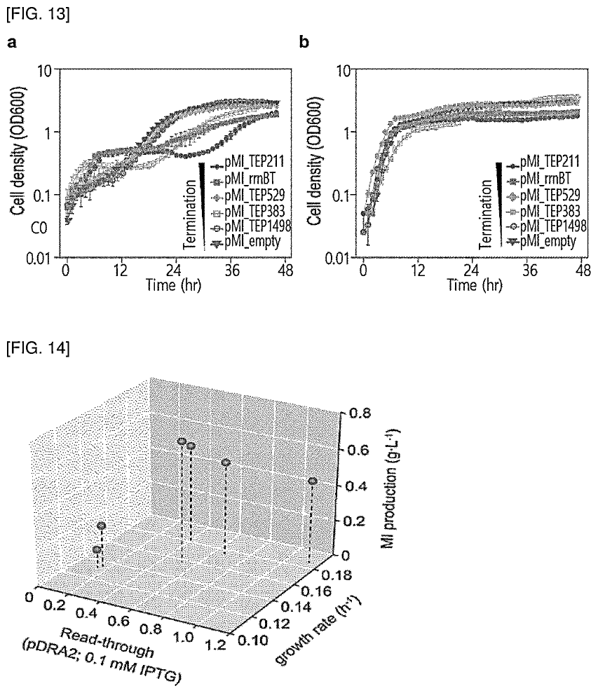

This flux regulation mechanism was used to control the flux between central carbon metabolism and myo-inositol (MI) production. When a biological system is used for production, the metabolic fluxes directed toward production and biomass must be balanced, as they are interconnected and constrained by a finite amount of cellular resources. MI-1-phosphate synthase (INO1) and phosphofructokinase I (PfkA, an effective rate-limiting enzyme of glycolysis) were combined as an interconnection point of production and cellular growth (FIG. 5D). An RIT located between the two genes acts as a metabolic valve to direct glycolytic resources (FIG. 5E). Five MI production plasmids encoding different valves were tested in glucose-6-phosphate 1-dehydrogenase (zwf) and pfkA double-knockout strains. Plasmids overexpressing INO1 without flux control displayed severe growth retardation and marginal MI production (FIG. 5F and FIG. 13). As pfkA expression increased in accordance with the increase in transcription read-through, glucose consumption and MI production also increased. At a read-through rate of 0.519 (T3PE-383), pfkA expression supported an MI production titer of 0.682 g/L, which was 8.92-times higher than that of the conventional system (pTrc-INO1) with a 38% MI yield (g/g) increase from glucose (FIG. 5F). When pfkA expression exceeded T3PE-383, the MI titer was reduced. At this point, excessive flux was used to promote cellular growth and biomass formation instead of MI production. Thus, an optimally rapid growth rate to support resources and energy for producing MI while not overconsuming a given nutrient could be successfully identified (FIG. 14). Then, the valves in genome-reduced E. coli that might have possessed a different optimum metabolic balance was examined. The genome-reduced strain reached its optimum metabolic balance at strong termination.

At optimal balance, where expression is terminated by a strong rrnB terminator, the production of 0.864 g/L of MI (1.27-fold higher than the maximum titer of MG1655) was identified. The MI titer was reduced after the plateau, where expression was higher with weaker terminators compared to that with rrnB terminator. This varying optimum metabolic balance indicates a lower carbon cost for the cellular growth of the genome-reduced strain compared to that for the growth of its wild-type counterpart (FIG. 13). These collective results demonstrate the novel use of transcription terminators in metabolic engineering. Terminators and metabolic valves can be operated in a scalable and reliable manner, regardless of the host strain. Sequences used in the experiments are shown in the following table.

TABLE 4
Sequence Listing

| | | |
|---|---|---|
| 1 | T3PE-211 | tcccgcaccattcaccagaaagcgttgtacggatggggtatcgccaagcggtaaggcaccggtttttgat |
| 2 | T3PE-529 | ctggttgaaaaagcgaaagcagctctggcataagccagttgaaagagggagctagtctccctcttttcgt |
| 3 | T3PE-383 | tttatgcgtgaactggaagagaacgcgatcgcttaatggtaaatcgggggcgtttctgcgcccccatctg |
| 4 | T3PE-1498 | gcaataaacttctggcagcgcgcattcagcagcagcaagatattgataacggaacgttgcctgattttat |
| 5 | T3PE-1345 | tggtgcaggcggcgcagaacttgcgtcgggggtaaaatccaaaccgggtggtaataccacccggtctttt |
| 6 | T3PE-936 | ctgcagtagcgatgctcgaagagcgcgctaagaagtaaaaaatcacagggcagggaaacctgcccttgtt |
| 7 | T3PE-390 | taaactgctgaaatgttcagagtttggtgacgcgatcatcgaaaacatgtaatgccgtagtttgttaaat |
| 8 | T3PE-948 | gctgcggatagtcgcagtggcattcttgcgttgcttaagcgcaccggttttcatctaccggtgtttttgt |
| 9 | T3PE-692 | aggtcagcggttcgatcccgcttagctccaccaaatttccaaccctcgctgcaaagcgggggttttttgt |
| 10 | T3PE-199 | cggcattccgaggttcgaatcctcgtaccccagccacattaaaaaagctcgcttcggcgagctttttgct |
| 11 | T3PE-455 | cgttttgttcagtgaatgatcttgccggatacacactgttcatagcctgcgccatacgcaggctatttct |
| 12 | rrnBT | caaataaaacgaaaggctcagtcgaaagactgggcctttcgttttatctgttgtttgtcggtgaacgctc |
| | | tcctgagtaggacaaat |
| 13 | aIsS | atgttgacaaaagcaacaaaagaacaaaaatcccttgtgaaaaacagaggggcggagcttgttgttgatt |
| | | gcttagtggagcaaggtgtcacacatgtatttggcattccaggtgcaaaaattgatgcggtatttgacgc |
| | | tttacaagataaaggacctgaaattatcgttgcccggcacgaacaaaacgcagcattcatggcccaagca |
| | | gtcggccgtttaactggaaaaccgggagtcgtgttagtcacatcaggaccgggtgcctctaacttggcaa |
| | | caggcctgctgacagcgaacactgaaggagaccctgtcgttgcgcttgctggaaacgtgatccgtgcaga |
| | | tcgtttaaaacggacacatcaatctttggataatgcggcgctattccagccgattacaaaatacagtgta |
| | | gaagttcaagatgtaaaaaatataccggaagctgttacaaatgcatttaggatagcgtcagcagggcagg |
| | | ctggggccgcttttgtgagctttccgcaagatgttgtgaatgaagtcacaaatacgaaaaacgtgcgtgc |
| | | tgttgcagcgccaaaactcggtcctgcagcagatgatgcaatcagtgcggccatagcaaaaatccaaaca |
| | | gcaaaacttcctgtcgttttggtcggcatgaaaggcggaagaccggaagcaattaaagcggttcgcaagc |
| | | ttttgaaaaaggttcagcttccatttgttgaaacatatcaagctgccggtaccctttctagagatttaga |
| | | ggatcaatattttggccgtatcggtttgttccgcaaccagcctggcgatttactgctagagcaggcagat |
| | | gttgttctgacgatcggctatgacccgattgaatatgatccgaaattctggaatatcaatggagaccgga |
| | | caattatccatttagacgagattatcgctgacattgatcatgcttaccagcctgatcttgaattgatcgg |
| | | tgacattccgtccacgatcaatcatatcgaacacgatgctgtgaaagtggaatttgcagagcgtgagcag |
| | | aaaatcctttctgatttaaaacaatatatgcatgaaggtgagcaggtgcctgcagattggaaatcagaca |
| | | gagcgcaccctcttgaaatcgttaaagagttgcgtaatgcagtcgatgatcatgttacagtaacttgcga |
| | | tatcggttcgcacgccatttggatgtcacgttatttccgcagctacgagccgttaacattaatgatcagt |
| | | aacggtatgcaaacactcggcgttgcgcttccttgggcaatcggcgcttcattggtgaaaccgggagaaa |
| | | aagtggtttctgtctctggtgacggcggtttcttattctcagcaatggaattagagacagcagttcgact |
| | | aaaagcaccaattgtacacattgtatggaacgacagcacatatgacatggttgcattccagcaattgaaa |
| | | aaatataaccgtacatctgcggtcgatttcggaaatatcgatatcgtgaaatatgcggaaagcttcggag |
| | | caactggcttgcgcgtagaatcaccagaccagctggcagatgttctgcgtcaaggcatgaacgctgaagg |
| | | tcctgtcatcatcgatgtcccggttgactacagtgataacattaatttagcaagtgacaagcttccgaaa |
| | | gaattcggggaactcatgaaaacgaaagctctctag |
| 14 | aIsD | atggaaactaatagctcgtgcgattgtgcaatcgaaatctcgcagcaatttgcgcgctggcaggcccgtc |
| | | aaggtgggggcgaggtctaccagtccagcctgatgtcggcactgctggcgggtgtttacgaaggcgaaac |
| | | cacaatggccgatctgctccgccacggggactttggtctgggcacgtttaaccggctggacggcgaactc |
| | | attgcctttgagcggcaaatccatcagttgaaagcggatggatctgcccgacccgctcgcgcagaacaga |
| | | aaacgccgtttgccgtgatgacgcacttccggccgtgcttgcaacgccggttcgctcatccgctgtcccg |
| | | cgaagaaattcaccaatgggtcgatcgcctcgtgggcactgacaacgttttcgttgcatttcgactggat |
| | | ggcttgtttgagcaagcgcaggtccgcaccgtcccctgtcagagcccaccctataagcccatgttggagg |
| | | ccattgaagcccagcctctgttcagtttcagtttgcggcgtgggaccctcgtcggctttcgctgcccacc |
| | | cttcgtgcaaggcattaacgtggctggctatcatgaacatttcattaccgaggatcgccgaggtgggggt |
| | | catatcttggattacgctatgggacacggccagctccaactgagcgtggttcaacacctcaacatcgagt |
| | | tgcctcgaaatcctgcctttcaacaggcagacctcaatccggcggatctggaccgcgctatccgtgcggc |
| | | tgagggttag |
| 15 | NO1 | atgacagaagataatattgctccaatcacctccgttaaagtagttaccgacaagtgcacgtacaaggaca |
| | | acgagctgctcaccaagtacagctacgaaaatgctgtagttacgaagacagctagtggccgcttcgatgt |
| | | aacgcccactgttcaagactacgtgttcaaacttgacttgaaaaagccggaaaaactaggaattatgctc |
| | | attgggttaggtggcaacaatggctccactttagtggcctcggtattggcgaataagcacaatgtggagt |
| | | ttcaaactaaggaaggcgttaagcaaccaaactacttcggctccatgactcaatgttctaccttgaaact |
| | | gggtatcgatgcggaggggaatgacgtttatgctccttttaactctctgttgcccatggttagcccaaac |
| | | gactttgtcgtctctggttgggacatcaataacgcagatctatacgaagctatgcagagaagtcaagttc |
| | | tcgaatatgatctgcaacaacgcttgaaggcgaagatgtccttggtgaagcctcttccttccatttacta |
| | | ccctgatttcattgcagctaatcaagatgagagagccaataactgcatcaatttggatgaaaaaggcaac |
| | | gtaaccacgaggggtaagtggacccatctgcaacgcatcagacgcgatatccagaatttcaaagaagaaa |
| | | acgcccttgataaagtaatcgttctttggactgcaaatactgagaggtacgtagaagtatctcctggtgt |
| | | taatgacaccatggaaaacctcttgcagtctattaagaatgaccatgaagagattgctccttccacgatc |
| | | tttgcagcagcatctatcttggaaggtgtcccctatattaatggttcaccgcagaatacttttgttcccg |
| | | gcttggttcagctggctgagcatgagggtacattcattgcgggagacgatctcaagtcgggacaaaccaa |
| | | gttgaagtctgttctggcccagttcttagtggatgcaggtattaaaccggtctccattgcatcctataac |
| | | catttaggcaataatgacggttataacttatctgctccaaaacaatttaggtctaaggagatttccaaaa |
| | | gttctgtcatagatgacatcatcgcgtctaatgatatcttgtacaatgataaactgggtaaaaaagttga |
| | | ccactgcattgtcatcaaatatatgaagcccgtcggggactcaaaagtggcaatggacgagtattacagt |
| | | gagttgatgttaggtggccataaccggatttccattcacaatgtttgcgaagattctttactggctacgc |
| | | ccttgatcatcgatcttttagtcatgactgagttttgtacaagagtgtcctataagaaggtggacccagt |
| | | taaagaagatgctggcaaattcgagaacttttatccagttttaaccttcttgagttactggttaaaagct |
| | | ccattaacaagaccaggatttcacccggtgaatggcttaaacaagcaaagaaccgccttagaaaattttt |
| | | taagattgttgattggattgccttctcaaaacgaactaagattcgaagagagattgttgtaa |
| 16 | bh | atggtgaaaggctttgcgatgctgagcataggcaaagtagggtggatcgaaaaagagaagccagcaccag |
| | | gtccgtttgatgcgattgttcggccactcgcggtcgctccgtgcacatctgacatccatacggtgttcga |
| | | gggagctattggggagcgccacaatatgatcctcggtcatgaggccgtcggcgaagttgttgaggtgggc |
| | | tccgaggtgaaggacttcaaaccgggagaccgggtggtggttccagcgattaccccggactggcggacgt |
| | | ctgaagttcagcgcggttatcaccagcacagcggtgggatgctggcgggctggaaatttagcaatgtgaa |
| | | agacggcgttttcggcgaattctttcatgttaatgatgcggacatgaatctggcccatctgcccaaggag |
| | | atccctctggaggccgccgtaatgattcccgacatgatgaccacgggattccacggcgccgaattagcag |
| | | acatcgaactgggtgcgacggtagcggttctcggtattggccccgtgggcctgatggctgtggcaggcgc |
| | | gaaactgcggggcgctggcaggattatcgcagtcgggagtcgccctgtttgtgtagatgctgccaagtac |
| | | tacggtgctaccgacattgtcaattataaggatggccccatcgagtcacagatcatgaatcttaccgagg |
| | | ggaaaggcgttgatgccgccatcatcgcagggggtaacgcggacattatggcaacagccgtcaaaattgt |
| | | gaaaccgggggggactatcgcgaacgtcaattatttcggcgagggcgaagtacttccggttccgcgtctc |
| | | gaatggggatgtggtatggctcacaaaacgattaagggtggcctttgcccaggaggacgcctgcggatgg |
| | | aacgcctgattgatctggtgttttacaagcgggtggatccatccaaactggtgacccacgtttttagagg |
| | | gtttgataatatcgagaaggcgttcatgctgatgaaggataaaccgaaagatctgattaaacctgtggtc |
| | | attctcgcttaa |
| 17 | pfkA | atgattaagaaaatcggtgtgttgacaagcggcggtgatgcgccaggcatgaacgccgcaattcgcgggg |
| | | ttgttcgttctgcgctgacagaaggtctggaagtaatgggtatttatgacggctatctgggtctgtatga |
| | | agaccgtatggtacagctagaccgttacagcgtgtctgacatgatcaaccgtggcggtacgttcctcggt |
| | | tctgcgcgtttcccggaattccgcgacgagaacatccgcgccgtggctatcgaaaacctgaaaaaacgtg |
| | | gtatcgacgcgctggtggttatcggcggtgacggttcctacatgggtgcaatgcgtctgaccgaaatggg |
| | | cttcccgtgcatcggtctgccgggcactatcgacaacgacatcaaaggcactgactacactatcggtttc |
| | | ttcactgcgctgagcaccgttgtagaagcgatcgaccgtctgcgtgacacctcttcttctcaccagcgta |
| | | tttccgtggtggaagtgatgggccgttattgtggagatctgacgttggctgcggccattgccggtggctg |
| | | tgaattcgttgtggttccggaagttgaattcagccgtgaagacctggtaaacgaaatcaaagcgggtatc |
| | | gcgaaaggtaaaaaacacgcgatcgtggcgattaccgaacatatgtgtgatgttgacgaactggcgcatt |
| | | tcatcgagaaagaaaccggtcgtgaaacccgcgcaactgtgctgggccacatccagcgcggtggttctcc |
| | | ggtgccttacgaccgtattctggcttcccgtatgggcgcttacgctatcgatctgctgctggcaggttac |
| | | ggcggtcgttgtgtaggtatccagaacgaacagctggttcaccacgacatcatcgacgctatcgaaaaca |
| | | tgaagcgtccgttcaaaggtgactggctggactgcgcgaaaaaactgtattaa |

While the present invention has been described with reference to the particular illustrative embodiments, a person skilled in the art to which the present invention pertains can understand that the present invention may be embodied in other specific forms without departing from the technical spirit or essential characteristics thereof. Therefore, the embodiments described above should be construed as being exemplified and not limiting the present invention. The scope of the invention should be construed to mean that the meaning and scope of the appended claims rather than the detailed description and all changes or variations derived from the equivalent concepts fall within the scope of the present invention.

1. A nucleic acid construct comprising first and second genes, of which expressions are regulated by one promoter, wherein the first gene is located upstream of the second gene, the promoter is located upstream of the first gene, and an E. coli-derived Rho-independent terminator is located at the 3′-end of the first gene.

2. The nucleic acid construct of claim 1, wherein the nucleic acid construct comprises a gene encoding an enzyme, which synthesizes acetoin from pyruvate, as the first gene and a gene encoding an enzyme, which synthesizes 2,3-butanediol from acetoin, as a second gene.

3. The nucleic acid construct of claim 2, wherein the first gene is composed of a gene encoding acetolactate synthase (AlsS) and acetolactate decarboxylase (AlsD) and the second gene is composed of a gene encoding butanediol dehydrogenase (Bdh).

4. The nucleic acid construct of claim 2, wherein the Rho-independent terminator of the nucleic acid construct is selected from SEQ ID NOS: 5 to 8.

5. The nucleic acid construct of claim 1, wherein the first gene comprised in the nucleic acid construct is a gene encoding MI-1-phosphate synthase (INO1) and the second gene comprised in the nucleic acid construct is a gene encoding phosphofructokinase I (PfkA).

6. The nucleic acid construct of claim 5, wherein the Rho-independent terminator of the nucleic acid construct is selected from SEQ ID NOS: 1 to 4.

7. A microorganism comprising the nucleic acid construct of claim 1.

8. A method for producing acetoin and BDO, the method comprising culturing a microorganism comprising the nucleic acid construct of claim 2.

9. A method for producing myo-inositol, the method comprising culturing a microorganism comprising the nucleic acid construct of claim 5.

10. The microorganism of claim 7, wherein the microorganism is Escherichia coli.


SEQUENCE LISTING (17 sequences)

SEQ ID NO: 1 - DNA, 70 bp, synthetic construct
  source 1..70: mol_type = other DNA; note = T3PE-211
tcccgcaccattcaccagaaagcgttgtacggatggggtatcgccaagcggtaaggcaccggtttttgat

SEQ ID NO: 2 - DNA, 70 bp, synthetic construct
  source 1..70: mol_type = other DNA; note = T3PE-529
ctggttgaaaaagcgaaagcagctctggcataagccagttgaaagagggagctagtctccctcttttcgt

SEQ ID NO: 3 - DNA, 70 bp, synthetic construct
  source 1..70: mol_type = other DNA; note = T3PE-383
tttatgcgtgaactggaagagaacgcgatcgcttaatggtaaatcgggggcgtttctgcgcccccatctg

SEQ ID NO: 4 - DNA, 70 bp, synthetic construct
  source 1..70: mol_type = other DNA; note = T3PE-1498
gcaataaacttctggcagcgcgcattcagcagcagcaagatattgataacggaacgttgcctgattttat

SEQ ID NO: 5 - DNA, 70 bp, synthetic construct
  source 1..70: mol_type = other DNA; note = T3PE-1345
tggtgcaggcggcgcagaacttgcgtcgggggtaaaatccaaaccgggtggtaataccacccggtctttt

SEQ ID NO: 6 - DNA, 70 bp, synthetic construct
  source 1..70: mol_type = other DNA; note = T3PE-936
ctgcagtagcgatgctcgaagagcgcgctaagaagtaaaaaatcacagggcagggaaacctgcccttgtt

SEQ ID NO: 7 - DNA, 70 bp, synthetic construct
  source 1..70: mol_type = other DNA; note = T3PE-390
taaactgctgaaatgttcagagtttggtgacgcgatcatcgaaaacatgtaatgccgtagtttgttaaat

SEQ ID NO: 8 - DNA, 70 bp, synthetic construct
  source 1..70: mol_type = other DNA; note = T3PE-948
gctgcggatagtcgcagtggcattcttgcgttgcttaagcgcaccggttttcatctaccggtgtttttgt

SEQ ID NO: 9 - DNA, 70 bp, synthetic construct
  source 1..70: mol_type = other DNA; note = T3PE-692
aggtcagcggttcgatcccgcttagctccaccaaatttccaaccctcgctgcaaagcgggggttttttgt

SEQ ID NO: 10 - DNA, 70 bp, synthetic construct
  source 1..70: mol_type = other DNA; note = T3PE-199
cggcattccgaggttcgaatcctcgtaccccagccacattaaaaaagctcgcttcggcgagctttttgct

SEQ ID NO: 11 - DNA, 70 bp, synthetic construct
  source 1..70: mol_type = other DNA; note = T3PE-455
cgttttgttcagtgaatgatcttgccggatacacactgttcatagcctgcgccatacgcaggctatttct

SEQ ID NO: 12 - DNA, 87 bp, synthetic construct
  source 1..87: mol_type = other DNA; note = rrnBT
caaataaaacgaaaggctcagtcgaaagactgggcctttcgttttatctgttgtttgtcggtgaacgctctcctgagtaggacaaat

SEQ ID NO: 13 - DNA, 1716 bp, Bacillus subtilis
  source 1..1716: mol_type = genomic DNA; note = alsS
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 14 - DNA, 780 bp, Aeromonas hydrophila
  source 1..780: mol_type = genomic DNA; note = alsD
atggaaactaatagctcgtgcgattgtgcaatcgaaatctcgcagcaatttgcgcgctggcaggcccgtcaaggtgggggcgaggtctaccagtccagcctgatgtcggcactgctggcgggtgtttacgaaggcgaaaccacaatggccgatctgctccgccacggggactttggtctgggcacgtttaaccggctggacggcgaactcattgcctttgagcggcaaatccatcagttgaaagcggatggatctgcccgacccgctcgcgcagaacagaaaacgccgtttgccgtgatgacgcacttccggccgtgcttgcaacgccggttcgctcatccgctgtcccgcgaagaaattcaccaatgggtcgatcgcctcgtgggcactgacaacgttttcgttgcatttcgactggatggcttgtttgagcaagcgcaggtccgcaccgtcccctgtcagagcccaccctataagcccatgttggaggccattgaagcccagcctctgttcagtttcagtttgcggcgtgggaccctcgtcggctttcgctgcccacccttcgtgcaaggcattaacgtggctggctatcatgaacatttcattaccgaggatcgccgaggtgggggtcatatcttggattacgctatgggacacggccagctccaactgagcgtggttcaacacctcaacatcgagttgcctcgaaatcctgcctttcaacaggcagacctcaatccggcggatctggaccgcgctatccgtgcggctgagggttag

SEQ ID NO: 15 - DNA, 1602 bp, Saccharomyces cerevisiae
  source 1..1602: mol_type = genomic DNA; note = ino1
atgacagaagataatattgctccaatcacctccgttaaagtagttaccgacaagtgcacgtacaaggacaacgagctgctcaccaagtacagctacgaaaatgctgtagttacgaagacagctagtggccgcttcgatgtaacgcccactgttcaagactacgtgttcaaacttgacttgaaaaagccggaaaaactaggaattatgctcattgggttaggtggcaacaatggctccactttagtggcctcggtattggcgaataagcacaatgtggagtttcaaactaaggaaggcgttaagcaaccaaactacttcggctccatgactcaatgttctaccttgaaactgggtatcgatgcggaggggaatgacgtttatgctccttttaactctctgttgcccatggttagcccaaacgactttgtcgtctctggttgggacatcaataacgcagatctatacgaagctatgcagagaagtcaagttctcgaatatgatctgcaacaacgcttgaaggcgaagatgtccttggtgaagcctcttccttccatttactaccctgatttcattgcagctaatcaagatgagagagccaataactgcatcaatttggatgaaaaaggcaacgtaaccacgaggggtaagtggacccatctgcaacgcatcagacgcgatatccagaatttcaaagaagaaaacgcccttgataaagtaatcgttctttggactgcaaatactgagaggtacgtagaagtatctcctggtgttaatgacaccatggaaaacctcttgcagtctattaagaatgaccatgaagagattgctccttccacgatctttgcagcagcatctatcttggaaggtgtcccctatattaatggttcaccgcagaatacttttgttcccggcttggttcagctggctgagcatgagggtacattcattgcgggagacgatctcaagtcgggacaaaccaagttgaagtctgttctggcccagttcttagtggatgcaggtattaaaccggtctccattgcatcctataaccatttaggcaataatgacggttataacttatctgctccaaaacaatttaggtctaaggagatttccaaaagttctgtcatagatgacatcatcgcgtctaatgatatcttgtacaatgataaactgggtaaaaaagttgaccactgcattgtcatcaaatatatgaagcccgtcggggactcaaaagtggcaatggacgagtattacagtgagttgatgttaggtggccataaccggatttccattcacaatgtttgcgaagattctttactggctacgcccttgatcatcgatcttttagtcatgactgagttttgtacaagagtgtcctataagaaggtggacccagttaaagaagatgctggcaaattcgagaacttttatccagttttaaccttcttgagttactggttaaaagctccattaacaagaccaggatttcacccggtgaatggcttaaacaagcaaagaaccgccttagaaaattttttaagattgttgattggattgccttctcaaaacgaactaagattcgaagagagattgttgtaa

SEQ ID NO: 16 - DNA, 1062 bp, Thermoanaerobacter brockii
  source 1..1062: mol_type = genomic DNA; note = bdh
atggtgaaaggctttgcgatgctgagcataggcaaagtagggtggatcgaaaaagagaagccagcaccaggtccgtttgatgcgattgttcggccactcgcggtcgctccgtgcacatctgacatccatacggtgttcgagggagctattggggagcgccacaatatgatcctcggtcatgaggccgtcggcgaagttgttgaggtgggctccgaggtgaaggacttcaaaccgggagaccgggtggtggttccagcgattaccccggactggcggacgtctgaagttcagcgcggttatcaccagcacagcggtgggatgctggcgggctggaaatttagcaatgtgaaagacggcgttttcggcgaattctttcatgttaatgatgcggacatgaatctggcccatctgcccaaggagatccctctggaggccgccgtaatgattcccgacatgatgaccacgggattccacggcgccgaattagcagacatcgaactgggtgcgacggtagcggttctcggtattggccccgtgggcctgatggctgtggcaggcgcgaaactgcggggcgctggcaggattatcgcagtcgggagtcgccctgtttgtgtagatgctgccaagtactacggtgctaccgacattgtcaattataaggatggccccatcgagtcacagatcatgaatcttaccgaggggaaaggcgttgatgccgccatcatcgcagggggtaacgcggacattatggcaacagccgtcaaaattgtgaaaccgggggggactatcgcgaacgtcaattatttcggcgagggcgaagtacttccggttccgcgtctcgaatggggatgtggtatggctcacaaaacgattaagggtggcctttgcccaggaggacgcctgcggatggaacgcctgattgatctggtgttttacaagcgggtggatccatccaaactggtgacccacgtttttagagggtttgataatatcgagaaggcgttcatgctgatgaaggataaaccgaaagatctgattaaacctgtggtcattctcgcttaa

SEQ ID NO: 17 - DNA, 963 bp, Escherichia coli
  source 1..963: mol_type = genomic DNA; note = pfkA
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa